(19) United States
(12) Patent Application Publication
(10) Pub. No.: US 2021/0161900 A1
(43) Pub. Date: Jun. 3, 2021

(54) COMPOSITIONS AND METHODS FOR THE TREATMENT OF SENESCENT TUMOR CELLS

(71) Applicant: Duke University, Durham, NC (US)

(72) Inventors: Xiao-Fan Wang, Durham, NC (US); Christopher Pan, Durham, NC (US); Lifeng Yuan, Durham, NC (US)

(21) Appl. No.: 17/047,258

(22) PCT Filed: Apr. 29, 2019
(86) PCT No.: PCT/US19/29573

§ 371 Date: Oct. 13, 2020

Related U.S. Application Data
(60) Provisional application No. 62/664,535, filed on Apr. 30, 2018.

(51) Int. Cl. A61K 31/519; A61P 35/04; A61K 45/06; A61K 31/506; A61K 31/443; A61K 31/4439; A61K 38/10; A61K 31/5377

(52) U.S. Cl. CPC A61K 31/519; A61P 35/04; A61K 45/06; A61K 31/5377; A61K 31/443; A61K 31/4439; A61K 38/10; A61K 31/506

(57) ABSTRACT

The present disclosure comprises compositions and methods for the treatment of senescent tumor cells. In particular, compositions and methods for countering negative effects of cancer therapy-induced senescence in tumor cells are provided.

### CROSS-REFERENCE TO RELATED APPLICATIONS

This application claims the priority benefit of U.S. Provisional Patent Application No. 62/664,535, filed Apr. 30, 2018, the entire contents of which are hereby fully incorporated by reference.

### STATEMENT OF GOVERNMENT INTEREST

This invention was made with Government support under Federal Grant No. CA154586 awarded by the NCI/NIH and W81XWH-16-1-0618 by the Department of Defense. The Federal Government has certain rights to this invention.

### TECHNICAL FIELD OF THE INVENTION

This invention is related to the area of ant-tumor therapy. In particular, the invention relates to decreasing senescence and senescence-associated secretory phenotype in cancer cells.

### BACKGROUND OF THE INVENTION

Cellular senescence refers to the irreversible arrest of cell proliferation that occurs when cells are repeatedly exposed to oncogenic stress. Cell senescence can suppress tumorigenesis through irreversible growth arrest. Treatment with anticancer drugs, such as palbociclib, results in cytostatic growth inhibition and senescence. However, despite their growth arrest, senescent cells remain metabolically active and secrete a number of proinflammatory chemokines/cytokines in a process known as senescence-associated secretory phenotype (SASP).

SASP has been implicated in therapy resistance and tumor recurrence. Given that senescent tumor cells can induce therapy resistance and tumor recurrence through SASP, it is critical to identify molecules that drive tumor cells towards apoptosis rather than senescence upon drug treatment.

### SUMMARY OF THE INVENTION

In one aspect, the invention relates to a method of treating a therapy-resistant cancer in a subject comprising administering to a subject a therapeutic amount of a protease-activated receptor (PAR) antagonist such that the therapy-resistant cancer is treated.

In one aspect, the invention relates to a method of treating a therapy-resistant cancer in a subject comprising administering to a subject a therapeutic amount of a protease-activated receptor (PAR) antagonist such that the therapy-resistant cancer is treated.

In one aspect, the invention relates to a method of treating a drug-induced, senescent cancer in a subject comprising administering to a subject a therapeutic amount of a protease-activated receptor (PAR) antagonist such that the drug-induced, senescent cancer is treated.

In one aspect, the invention relates to a method of treating a therapy-resistant cancer in a subject comprising administering to a subject a therapeutic amount of a thrombin inhibitor, such that the therapy-resistant cancer is treated.

In one aspect, the invention relates to a method of treating a drug-induced, senescent cancer in a subject comprising administering to a subject a therapeutic amount of a thrombin inhibitor, such that the drug-induced, senescent cancer is treated.

In one aspect, the invention relates to a method of inducing apoptosis in a senescent tumor cell comprising administering to a subject an effective amount of a protease-activated receptor (PAR) antagonist or a thrombin inhibitor, such that apoptosis is induced in the tumor cell.

In one aspect, the invention relates to a method of inducing apoptosis in a senescent tumor cell comprising contacting the senescent tumor cell with a protease-activated receptor (PAR) antagonist or a thrombin inhibitor, such that apoptosis is induced in the tumor cell.

### BRIEF DESCRIPTION OF THE DRAWINGS

FIG. 1 is a schematic model depicting the therapeutic application of combinational therapy of THBD signaling inhibition and palbociclib in breast cancer and NSCLC.

FIG. 2 is a schematic representation of proposed mechanism for THBD signaling-mediated cell fate. THBD facilitates PC-mediated, N-terminal PAR1 cleavage at R46, resulting in downstream G12/13-mediated signaling, apoptosis suppression, and senescence induction.

FIGS. 3A-30 show that THBD is upregulated in various cell lines by different senescent stimuli and in aged tissues: FIG. 3A shows THBD mRNA and FIG. 3B shows protein expression in NHBE cells treated with DMSO or 1 μM erlotinib for 24 and 48 h; FIGS. 3C and 3D show THBD protein expression in HBE cells undergoing oncogene- and replicative-induced senescence, respectively; FIG. 3E shows THBD mRNA expression in HBE cells undergoing replicative senescence; FIGS. 3F and 3G show THBD protein expression in IMR-90 cells undergoing oncogene- and replicative-induced senescence, respectively; FIGS. 3H and 31 show THBD mRNA expression in IMR-90 cells undergoing oncogene- and replicative-induced senescence, respectively; FIGS. 3J-3M show THBD protein expression in young and aged lung, heart, muscle, and liver tissues; respectively; FIG. 3N shows THBD mRNA expression in young and aged lung tissues; and FIG. 3M shows THBD mRNA expression in young and aged liver tissues.

FIGS. 4A-4F show that THBD signaling is upregulated by multiple senescent stimuli and in aged tissue: FIGS. 4A and 4B show Western analysis of THBD signaling components, PAR1, protein C, thrombin, EPCR, and SERPINA5, in IMR-90 cells undergoing oncogene-induced and replicative senescence, respectively; FIGS. 4C-4F show Western analysis of THBD signaling components, PAR1, protein C, thrombin, Gal2, Gal3, and RhoA, in young and aged lung, heart, muscle, and liver tissues, respectively.

FIGS. 5A-5C show that THBD-signaling pathway components are upregulated at different stages of senescence: FIG. 5A shows IMR-90 cells stably expressing HRas under the control of doxycycline (DOX), treated with doxycycline for either 1, 2, 3, 4, 5, 6 or 7 days and immunoblotted for THBD-signaling pathway components; FIG. 5B shows HBE cells treated with erlotinib (1 μM) for either 1, 2, 3, 4, 5, 6, 7, or 8 days and immunoblotted for THBD-signaling pathway components; and FIG. 5C shows IMR-90 cells stably expressing control (NTC) or THBD-targeting shRNA (shTHBD) immunoblotted for THBD-signaling pathway components.

FIGS. 6A-6C show that THBD signaling is required for senescence-mediated growth arrest and SASP production: FIG. 6A shows representative brightfield and SA-βGal images; FIG. 6B shows Western analysis of senescence markers, pRB, p21, p16; and FIG. 6C shows Western analysis of SASP mediators, p-NFkB p65, IL-8, and IL-6 in IMR-90 cells stably expressing a control shRNA or THBD-targeting shRNA undergoing HRas-induced senescence.

FIGS. 7A-7C show that THBD signaling determines whether cells undergo senescence or apoptosis during HRas stimulation and is critical for HRas-induced senescent cell survival: FIGS. 7A and 7B show Western analysis of senescent and apoptotic markers in IMR-90 and NHBE cells, respectively, each transiently infected with control shRNA or THBD-targeting shRNA followed by treatment with doxycycline for 7 days to induce HRas expression; and FIG. 7C shows Western analysis of cleaved caspase-3 in HRas-induced senescent IMR-90 cells (proliferating IMR-90 cells were treated with doxycycline for 7 days to induce senescence—following senescence establishment, cells were infected with shRNA or THBD-targeting shRNA).

FIGS. 8A-8D shows that palbociclib induces senescence in multiple breast cancer subtypes: FIG. 8A shows representative images of SA-βGal staining of MCF7, MDA-MB-231, and AU565 cells treated with indicated doses of palbociclib for 7 days; and FIGS. 8B-8D show Western analysis of senescent markers, pRB and p21, in MCF7, MDA-MB-231, and AU565 cells, respectively, each treated with increasing doses (0.1 μM, 0.5 μM, 1 μM) of palbociclib for 7 days.

FIGS. 9A-9C show that THBD signaling is upregulated during palbociclib-induced senescence: FIGS. 9A-9C show western analysis of THBD signaling components, THBD, PAR1, Protein C, and thrombin, in MCF7, MDA-MB-231, and AU565 cells, respectively, each treated with increasing doses (0.1 μM, 0.5 μM, 1 μM) of palbociclib for 7 days.

FIGS. 10A and 10B show that THBD signaling determines whether MCF7 undergo senescence or apoptosis during palbociclib treatment and is critical for palbociclib-induced senescent cell survival: FIG. 10A shows western analysis of senescent and apoptotic markers in MCF7 cell stably infected with control shRNA (NTC) or THBD-targeting shRNA followed by treatment with increasing doses of (0.1 μM, 0.5 μM, 1 μM) palbociclib for 7 days; and FIG. 10B shows western analysis of cleaved caspase-7 in palbociclib-induced senescent MCF7 (MCF7 cells were treated with increasing doses (0.1 μM, 0.5 μM, 1 μM) of palbociclib for 7 days—following senescence establishment, MCF7 cells were infected with NTC or THBD-targeting shRNA).

FIGS. 11A and 11B are schematic representations of THBD-signaling pathway and inhibitory actions of vorapaxar and dabigatran: FIG. 11A illustrates that THBD binds to thrombin and places thrombin in close proximity to PC, thrombin cleaves and activates PC (aPC), aPC proteolytically cleaves PAR1 generating a N-terminus ligand that binds and activates PAR1, resulting in senescence establishment and apoptosis suppression; and FIG. 11B illustrates that dabigatran competitively binds to thrombin and acts as a direct-thrombin inhibitor and that vorapaxar blocks proteolytical cleavage of PAR1, resulting in senescence suppression and apoptosis induction.

FIG. 12 shows that PAR1 regulates MDA-MB-231 cell fate. MDA-MB-231 cells were cotreated with palbociclib (0.5 μM) and vorapaxar (10 μM) or SCH79797 (100 nM) for 7 days and immunoblotted for apoptotic marker, cleaved caspase-3.

FIGS. 13A-13C show that PAR1 is a viable senolytic target and vorapaxar is a novel senolytic agent: FIG. 13A shows western analysis of cleaved caspase-7 in palbociclib-induced senescent MCF7 cells treated with increasing doses (25 nM, 50 nM, 75 nM, 150 nM, 500 nM) of SCH79797 for 24 h; FIGS. 13B and 13C show western analysis of cleaved caspase-3 in palbociclib-induced senescent MDA-MB-231 cells treated with increasing doses (50 nM, 100 nM, 200 nM, 500 nM, 1 μM) of vorapaxar (FIG. 13B) and (25 nM, 75 nM, 150 nM, 500 nM) of SCH79797 (FIG. 13C) for 24 h.

FIG. 14 is a schematic representation of PAR1-mediated G-protein signaling. PAR1 couples to the heterotrimeric G proteins G12/13, Gq, and Gi to activate multiple signaling effectors. Inhibition of G12/13, Gq, and Gi signaling can be achieved through overexpressing regulators of G-protein signaling (RGS proteins) or using pharmacological inhibitors such as C3 transferase and Y-27632.

FIGS. 15A-15C show that G12/13 is critical for MDA-MB-231 senescent cell survival: FIG. 15A shows western analysis for control and palbociclib-induced senescent MDA-MB-231 cells were infected with NTC, G12/13 inhibitor, p115-RGS, and Gq inhibitor, RGS2, and immunoblotted for cleaved caspase-3; and FIGS. 15B and 15C show western analysis for control and senescent MDA-MB-231 cells were treated with increasing doses of RhoA inhibitor, C3 transferase (FIG. 15B), and ROCK-1 inhibitor, Y-27632 (FIG. 15C), and immunoblotted for cleaved caspase-3.

FIGS. 16A-16D show that palbociclib induces senescence in multiple NSCLC cell lines: FIG. 16A shows representative images of SA-βGal staining of HCC827, H1650, and PC9 cells treated with indicated doses of palbociclib for 7 days; FIGS. 16B-16D show western analysis of senescent markers, pRB and p21, in HCC827, H1650, and PC9 cells, respectively, each treated with increases doses (0.1 μM, 0.5 μM, 1 μM) of palbociclib for 7 days.

FIGS. 17A and 17B show that THBD signaling is upregulated during palbociclib-induced senescence: FIGS. 17A and 17B show western analysis of THBD signaling components, THBD, PAR1, Protein C, and thrombin, in HCC827 and HCC1650, respectively, each treated with increasing doses (0.1 μM, 0.5 μM, 1 μM) of palbociclib for 7 days.

FIG. 18 shows that THBD signaling regulates NSCLC cell fate. HCC827 cells were transiently infected with NTC or shRNAs targeting THBD. Following infection, cells were treated with increasing doses of palbociclib (0.1 μM, 0.5 μM, 1 μM) for 7 days and immunoblotted for senescent and apoptotic markers.

FIGS. 19A-19E show that PAR1 inhibition promotes apoptosis in palbociclib-induced senescent NSCLC cells: FIGS. 19A-19C show western analysis of H1650 (FIG. 19A) and HCC827 cells (FIG. 19B), each treated with palbociclib (0.5 μM) for 7 days and treated with increasing doses of vorapaxar (25 nM, 50 NM, 100 nM, 500 nM, 1 μM) or SCH79797 (25 nM, 50 nM, 75 nM, 150 nM, 500 nM) (FIG. 19C) and immunoblotted for cleaved caspase-3; and FIGS. 19D and 19E show representative brightfield images of HCC827 cells treated with indicated doses of vorapaxar and SCH79797, respectively, after palbociclib treatment.

FIG. 20 shows that PAR1 regulates NSCLC cell fate. NCLC cells were cotreated with palbociclib (0.5 μM) and vorapaxar (10 μM) or SCH79797 (100 nM) for 7 days and immunoblotted for apoptotic marker, cleaved caspase-3.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising/* or “having” certain elements are also contemplated as “consisting essentially of and “consisting of those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification.

These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

“Treatment,” “therapy” and/or “therapy regimen” refer to the clinical intervention made in response to a disease, disorder, or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition.

The term “effective amount” or “therapeutically effective amount” refers to an amount sufficient to effect beneficial or desirable biological and/or clinical results. The term “subject” and “patient” are used interchangeably herein and refer to both human and nonhuman animals. The term “nonhuman animals” includes all vertebrates, e.g., mammals and non-mammals, such as nonhuman primates, sheep, dog, cat, horse, cow, chickens, amphibians, reptiles, and the like. Preferably, the subject is a human patient is suffering from, or at risk of developing, cancer. The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth (e.g., tumor cell). The methods of the present disclosure can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In some embodiments, the cancer comprises pancreatic cancer.

In one aspect, the invention relates to a method of treating a therapy-resistant cancer in a subject comprising administering to a subject a therapeutic amount of a protease-activated receptor (PAR) antagonist such that the therapy-resistant cancer is treated.

In one aspect, the invention relates to a method of treating a therapy-resistant cancer in a subject comprising administering to a subject a therapeutic amount of a protease-activated receptor (PAR) antagonist such that the therapy-resistant cancer is treated.

In one aspect, the invention relates to a method of treating a drug-induced, senescent cancer in a subject comprising administering to a subject a therapeutic amount of a protease-activated receptor (PAR) antagonist such that the drug-induced, senescent cancer is treated.

In some embodiments, the protease-activated receptor (PAR) antagonist is administered concurrently with one or more anti-cancer drugs.

In some embodiments, the protease-activated receptor (PAR) antagonist is administered prior to the administration of one or more anti-cancer drugs. In some embodiments, the protease-activated receptor (PAR) antagonist is administered after the administration of one or more anti-cancer drugs.

In some embodiments, the subject has been treated with a therapy known to induce senescence. In some embodiments, the therapy is CDK 4/6 inhibitors or DNA-damaging agents. In some embodiments, the therapy is treatment with CDK 4/6 inhibitors.

In some embodiments, the one or more anti-cancer drugs is palbociclib, ribociclib, or abemaciclib. In some embodiments, the anti-cancer drug is palbociclib.

In some embodiments, the protease-activated receptor (PAR) antagonist is a selective antagonist of protease activated receptor 1 (PAR1). In some embodiments, the protease-activated receptor (PAR) antagonist is vorapaxar (SCH 530348), SCH 79797, atopaxar (E5555), any derivatives, esters and salts thereof, or combinations thereof. In some embodiments, the protease-activated receptor (PAR) antagonist is vorapaxar. In some embodiments, the vorapaxar is administered at a dosage of from about 0.03 mg/kg to about 15 mg/kg, or from about 0.5 mg/kg to about 10 mg/kg. In some embodiments, the dosage is about 0.5 mg/kg, about 1 mg/kg, or about 10 mg/kg.

In some embodiments, the cancer is breast cancer. In some embodiments, the breast cancer is metastatic ER+, HER2− breast cancer. In some embodiments, the breast cancer is a HER2+ breast cancer. In some embodiments, the breast cancer is triple-negative breast cancer.

In some embodiments, the cancer is lung cancer. In some embodiments, the lung cancer is non-small cell lung cancer.

In one aspect, the invention relates to a method of treating a therapy-resistant cancer in a subject comprising administering to a subject a therapeutic amount of a thrombin inhibitor, such that the therapy-resistant cancer is treated.

In one aspect, the invention relates to a method of treating a drug-induced, senescent cancer in a subject comprising administering to a subject a therapeutic amount of a thrombin inhibitor, such that the drug-induced, senescent cancer is treated.

In some embodiments, the subject also suffers from cancer-associated thrombosis.

In some embodiments, the thrombin inhibitor is administered concurrently with one or more anti-cancer drugs. In some embodiments, the thrombin inhibitor is administered prior to the administration of one or more anti-cancer drugs. In some embodiments, the thrombin inhibitor is administered after the administration of one or more anti-cancer drugs.

In some embodiments, the subject has been treated with a therapy known to induce senescence. In some embodiments, the therapy is CDK 4/6 inhibitors or DNA damaging agents.

In some embodiments, the therapy is an anti-cancer drug. In some embodiments, the anti-cancer drug is selected from the group consisting of palbociclib, doxorubicin, and cisplatin. In some embodiments, the anti-cancer drug is palbociclib.

In some embodiments, the thrombin inhibitor is dabigatran, lepirudin, desirudin, bivalirudin, argatroban, any derivatives, esters and salts thereof, or combinations thereof. In some embodiments, the thrombin inhibitor is dabigatran. In some embodiments, the dabigatran is administered in a dosage of from about 18 mg/kg to about 120 mg/kg, about 18 mg/kg, about 37.5 mg/kg, about 75 mg/kg, or about 120 mg/kg. In some embodiments, the thrombin inhibitor is bivalirudin. In some embodiments, the bivalirudin is administered in a dosage of from about 18 mg/kg to about 120 mg/kg. In some embodiments, the dosage is about 18 mg/kg, about 37.5 mg/kg, about 75 mg/kg, or about 120 mg/kg.

In one aspect, the invention relates to a method of inducing apoptosis in a senescent tumor cell comprising administering to a subject an effective amount of a protease-activated receptor (PAR) antagonist or a thrombin inhibitor, such that apoptosis is induced in the tumor cell.

In some embodiments, a protease-activated receptor (PAR) antagonist is administered. In some embodiments, the protease-activated receptor (PAR) antagonist is selective for PAR1. In some embodiments, the protease-activated receptor (PAR) antagonist is vorapaxar (SCH 530348), SCH 79797, atopaxar (E5555), any derivatives, esters and salts thereof, or combinations thereof. In some embodiments, the protease-activated receptor (PAR) antagonist is vorapaxar.

In some embodiments, a thrombin inhibitor is administered. In some embodiments, the thrombin inhibitor is dabigatran, lepirudin, desirudin, bivalirudin, argatroban, any derivatives, esters and salts thereof, or combinations thereof. In some embodiments, the thrombin inhibitor is dabigatran.

In one aspect, the invention relates to a method of inducing apoptosis in a senescent tumor cell comprising contacting the senescent tumor cell with a protease-activated receptor (PAR) antagonist or a thrombin inhibitor, such that apoptosis is induced in the tumor cell.

In some embodiments, the cell is contacted with a protease-activated receptor (PAR) antagonist. In some embodiments, the protease-activated receptor (PAR) antagonist is vorapaxar (SCH 530348), SCH 79797, atopaxar (E5555), any derivatives, esters and salts thereof, or combinations thereof. In some embodiments, the protease-activated receptor (PAR) antagonist is selective for PAR1. In some embodiments, the protease-activated receptor (PAR) antagonist is vorapaxar.

In some embodiments, the cell is contacted with a thrombin inhibitor. In some embodiments, the thrombin inhibitor is dabigatran, lepirudin, desirudin, bivalirudin, argatroban, any derivatives, esters and salts thereof, or combinations thereof. In some embodiments, the thrombin inhibitor is dabigatran.

In some embodiments, the cell is characterized therapy-induced senescence. In some embodiments, the therapy is CDK 4/6 inhibitors or DNA damaging agents. In some embodiments, the therapy is CDK 4/6 inhibition.

In some embodiments, the therapy is an anti-cancer drug. In some embodiments, the anti-cancer drug is palbociclib, ribociclib, or abemaciclib. In some embodiments, the anti-cancer drug is palbociclib.

### EXAMPLES

The following Examples are provided by way of illustration and not by way of limitation.

Breast Cancer

Palbociclib (PD-03329, trade name Ibrance®, Pfizer) is the first CDK4/6 inhibitor to be approved for cancer therapy and is currently used in combination with the aromatase inhibitor, letrozole, for the treatment of ER+ and HER2-metastatic breast cancer (Kim, E. S. and L. J. Scott, Palbociclib: A Review in HR-Positive, HER2-Negative, Advanced or Metastatic Breast Cancer. Target Oncol, 2017. 12(3): p. 373-383). While initially effective, nearly half of palbociclib-treated patients experience adverse side effects or develop resistance and disease progression after two years of treatment (Lim, S. and P. Kaldis, Cdks, cyclins and CKIs: roles beyond cell cycle regulation. Development, 2013. 140(15): p. 3079-93; Trere, D., et al., High prevalence of retinoblastoma protein loss in triple-negative breast cancers and its association with a good prognosis in patients treated with adjuvant chemotherapy. Ann Oncol, 2009. 20(11): p. 1818-23; Herrera-Abreu, M. T., et al., Early Adaptation and Acquired Resistance to CDK4/6 Inhibition in Estrogen Receptor-Positive Breast Cancer. Cancer Res, 2016. 76(8): p. 2301-13; Yang, C., et al., Acquired CDK6 amplification promotes breast cancer resistance to CDK4/6 inhibitors and loss of ER signaling and dependence. Oncogene, 2017. 36(16): p. 2255-2264).

Palbociclib exerts its therapeutic effects by inducing cellular senescence, a state of irreversible cell-cycle arrest. Despite being growth arrested, senescent cells remain metabolically active and can create a pro-tumorigenic microenvironment, resulting in therapy resistance and eventual disease recurrence. Based on initial findings, it was believed that disruption of the senescence program induced by palbociclib could lead to a change in cell fate from senescence to apoptosis in breast cancer cells. Thus, the use of senolytic therapies to promote synthetic lethality may bypass the negative side effects of senescence and enhance the efficacy of palbociclib by driving palbociclib-treated cells towards apoptosis rather than senescence.

Through genetic screening, thrombomodulin (THBD) was identified as a novel senolytic target for palbociclib-induced senescence. THBD-mediated signaling was up-regulated during palbociclib-induced senescence in ER+, HER2+, and triple negative breast cancer cell lines and served as a critical regulator of breast cancer cell fate and survival as depletion of THBD in breast cancer cells attenuated senescence and promoted apoptosis upon palbociclib treatment. Importantly, inhibiting the activity of PAR1, a THBD downstream effector, by an FDA-approved drug, vorapaxar, caused senescent breast cancer cells to apoptose under treatment of palbociclib. Taken together, these results reveal that THBD-mediated signaling regulates breast cancer cell fate and survival and provide the molecular basis use of this pathway to induce synthetic lethality in palbociclib-treated breast cancer cells.

The methods herein can reduce or eliminate the mortality associated with metastatic breast cancer by promoting apoptosis of senescent cancer cells and leading to a significant reduction in the mortality associated with metastasis of those patients.

THBD signaling is required for palbociclib-induced senescent cell survival and is an important determinant of whether breast cancer cells undergo senescence or apoptosis in response to palbociclib. The disruption of THBD signaling can benefit palbociclib-treated patients due to the induction of apoptosis for breast cancer cells.

To summarize, THBD has been identified as a novel regulator of senescence. THBD signaling was up-regulated by multiple senescent stimuli in epithelial cells and fibroblasts. Functionally, THBD signaling mediated cell fate determination during oncogenic stress and promoted senescent cell viability. Importantly, these findings were also observed in palbociclib-treated breast cancer cells. THBD signaling was elevated in palbociclib-induced senescent cells, promoted the induction and establishment of palbociclib-mediated senescence, and served as a pro-survival factor for palbociclib-induced senescent cells. Overall, these results indicate that inhibition of THBD signaling in combination with palbociclib is a promising therapeutic strategy for attenuating senescence and promoting synthetic lethality in a broad-range of breast cancer subtypes.

### Example 1—Palbociclib Induces Senescence in Multiple Breast Cancer Subtypes

Although palbociclib is currently FDA-approved for the treatment of only metastatic ER+ and HER2− breast cancer, the nature of its cell cycle inhibition and senescence-induction suggests that it may have therapeutic potential in a broad-range of breast cancer subtypes (Rocca, A., et al., Progress with palbociclib in breast cancer: latest evidence and clinical considerations. Ther Adv Med Oncol, 2017. 9(2): p. 83-105; Valenzuela, C. A., et al., Palbociclib-induced autophagy and senescence in gastric cancer cells. Exp Cell Res, 2017. 360(2): p. 390-396; Vijayaraghavan, S., et al., CDK4/6 and autophagy inhibitors synergistically induce senescence in Rb positive cytoplasmic cyclin E negative cancers. Nat Commun, 2017. 8: p. 15916). Therefore, these studies sought to investigate whether palbociclib-induces senescence in other types of breast cancer, including HER2+ and triple-negative breast cancer. Using an ER+, HER2− cell line, MCF7, as a control, a HER2+ cell line, AU565, and a triple-negative cell line, MDA-MB-231, were treated with increasing doses of palbociclib.

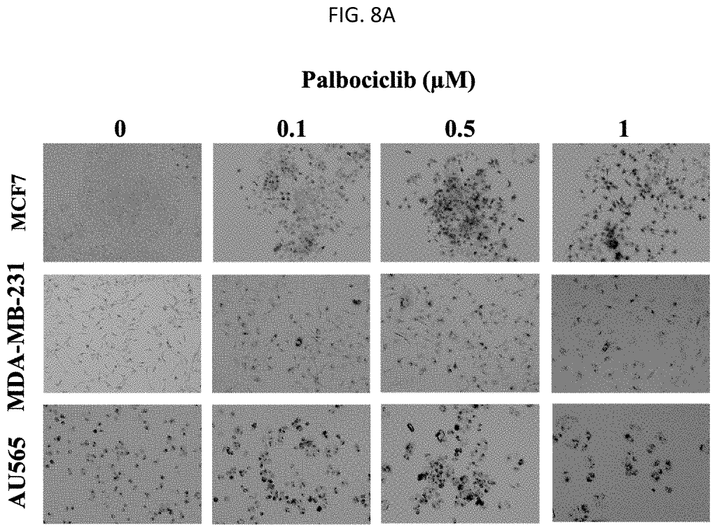

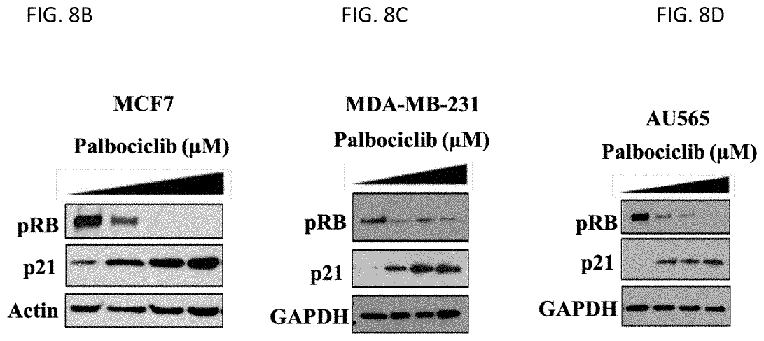

Palbociclib-treated cells exhibited cytostatic inhibition and appeared larger and flatter compared to non-treated cells, suggesting that palbociclib could induce cellular senescence to promote growth arrest. Molecularly, senescent cells exhibit a common set of characteristics that include hypophosphorylation of Rb, upregulation of cyclin-dependent kinase inhibitors, p16 and p21, and increased expression of the lysosomal enzyme, β-galactosidase (senescence-associated β-galactosidase, SA-βgal) (Campisi, J., Aging, cellular senescence, and cancer. Annu Rev Physiol, 2013. 75: p. 685-705; Carnero, A., Markers of cellular senescence. Methods Mol Biol, 2013. 965: p. 63-81). To determine whether cellular senescence might account for the growth inhibition following palbociclib treatment, non-treated and treated cells were stained for SA-0 gal and, in parallel, immunoblotted for pRB, p21, and p16. Palbociclib-treated cells exhibited robust SA-0 gal staining and a dose-dependent increase in p21, p16, and Rb hypophosphorylation (FIGS. 8A-8D). These results indicate that palbociclib potently inhibits the growth of multiple types of breast cancer cells by inducing cellular senescence.

### Example 2—THBD Signaling is Elevated in Multiple Senescent Contexts

It was demonstrated previously that inhibition of epidermal growth factor receptor (EGFR) through erlotinib treatment is sufficient to induce senescence in primary human bronchial epithelial (HBE) cells (Alexander, P. B., et al., EGF promotes mammalian cell growth by suppressing cellular senescence. Cell Res, 2015. 25(1): p. 135-8). Based on this observation, an unbiased gene expression profiling approach was developed to identify novel drivers of senescence by comparing genes significantly altered in senescent HBE cells versus their proliferating counterparts (Yuan, L., et al., Switching off IMMP2 L signaling drives senescence via simultaneous metabolic alteration and blockage of cell death. Cell Res, 2018. 28(6): p. 625-643). Of the candidates, THBD was one of the most upregulated genes in senescent cells, exhibiting a four-fold increase.

THBD is a type 1 transmembrane receptor that is primarily found on endothelial cells Martin, F. A., et al., Thrombomodulin and the vascular endothelium: insights into functional, regulatory, and therapeutic aspects. Am J Physiol Heart Circ Physiol, 2013. 304(12): p. H1585-97; Cheng, Y., et al., Intraovarian thrombin and activated protein C signaling system regulates steroidogenesis during the periovulatory period. Mol Endocrinol, 2012. 26(2): p. 331-40). Its archetypical function is to attenuate the pro-coagulant functions of thrombin and shift its specificity towards the protein C (PC) pathway. THBD-mediated binding of thrombin results in the activation of PC, which proteolytically activates the G-protein-coupled receptor, protease-activated receptor (PAR1), to elicit downstream signaling (Okamoto, T., et al., Thrombomodulin: a bifunctional modulator of inflammation and coagulation in sepsis. Crit Care Res Pract, 2012. 2012: p. 614545). While the role of THBD in coagulation is well-documented, its role in senescence has been largely unknown.

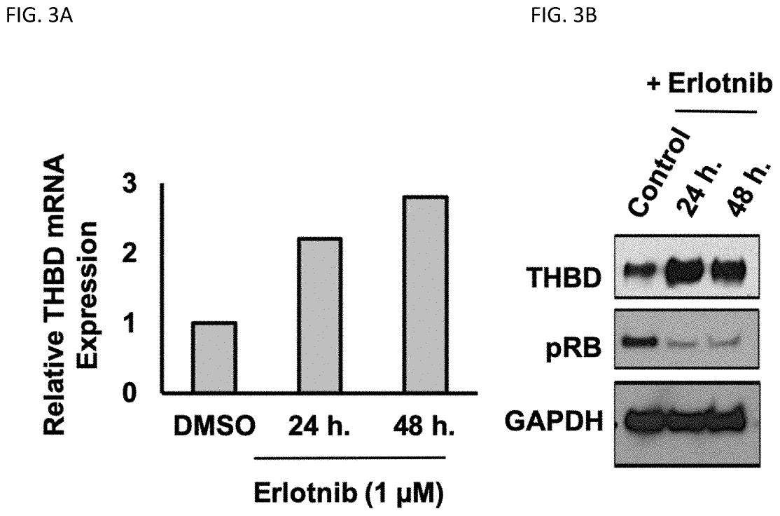

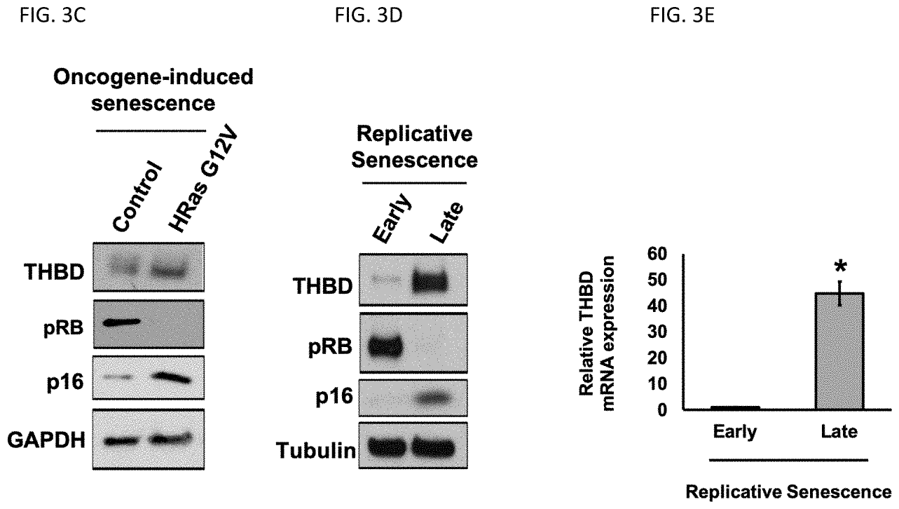

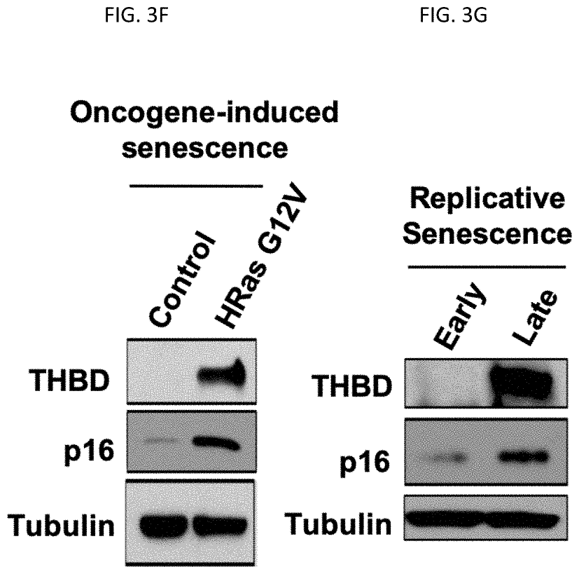

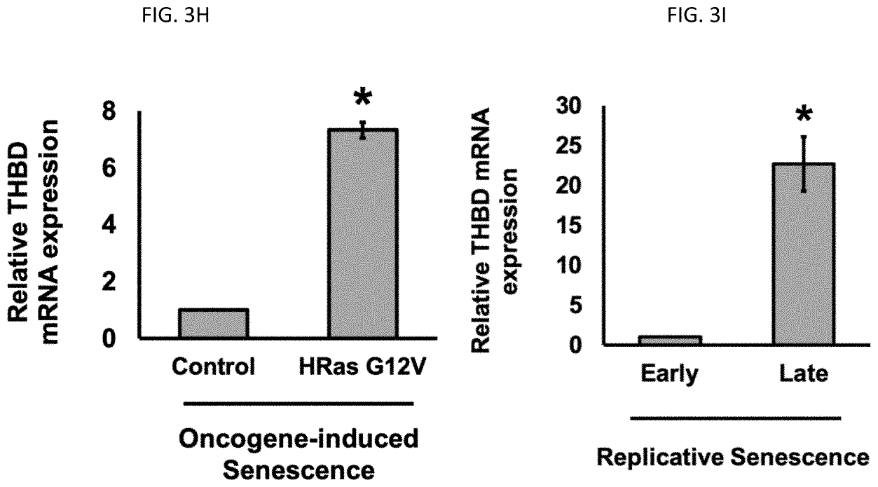

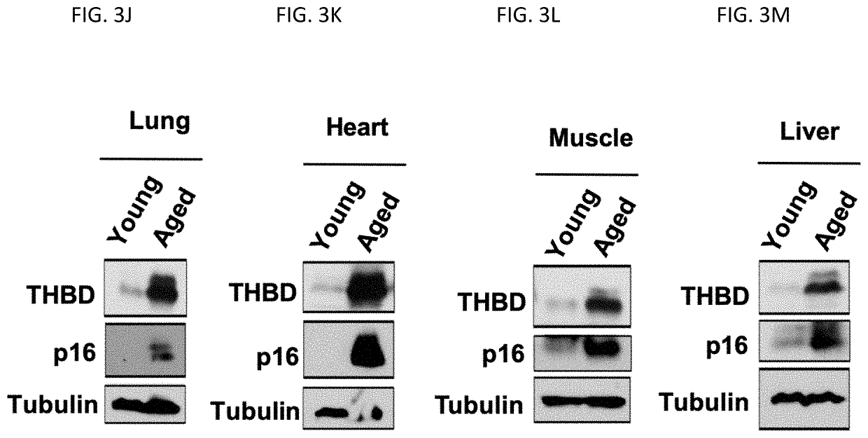

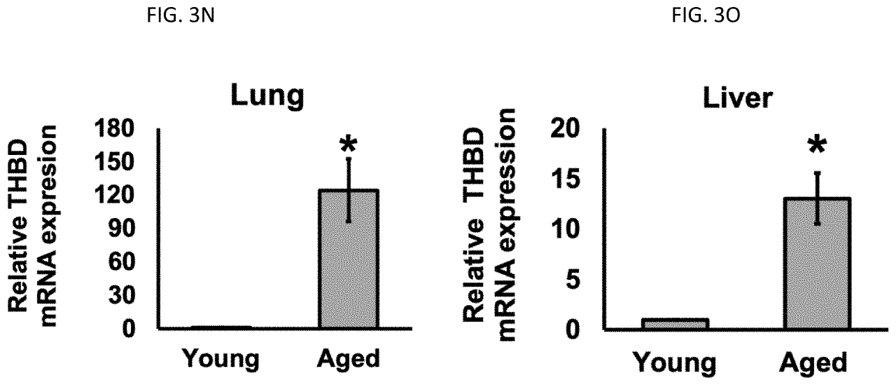

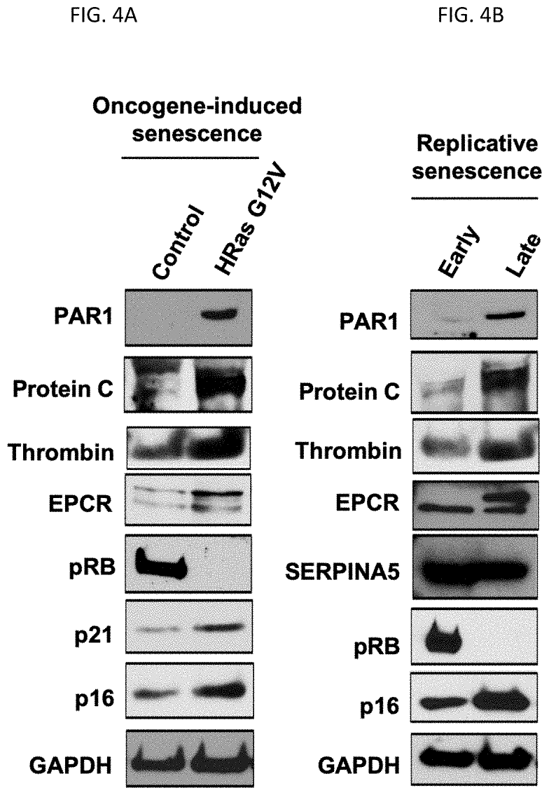

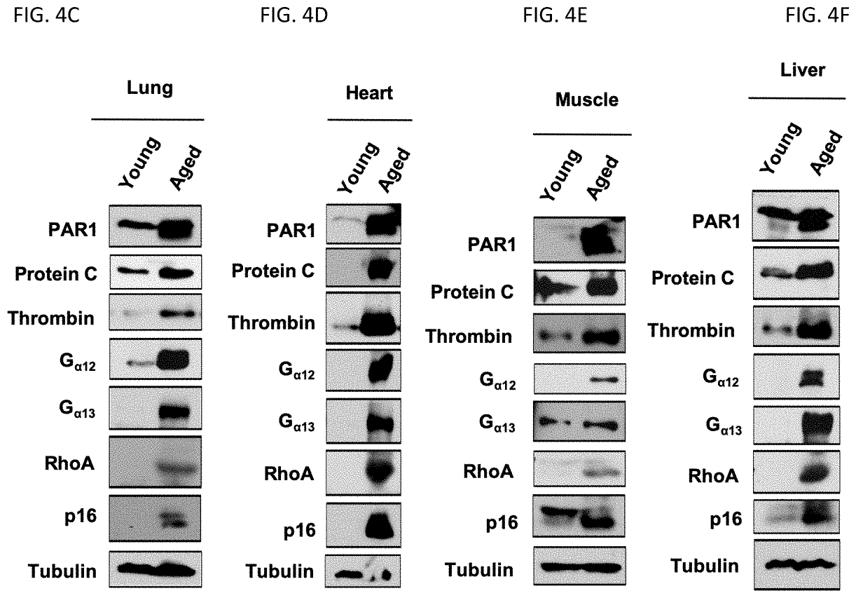

To validate gene profiling results, experiments were performed to determine whether THBD was up-regulated in erlotinib-induced, oncogene-induced, and replicative senescence in IMR-90, a cell line that is commonly used for senescence studies, and HBE cells. As shown in FIGS. 3B-3D, 3F and 3G, THBD was up-regulated in all senescence contexts in both cell types. As previously mentioned, THBD primarily signals through the thrombin-PC-PAR1 axis to elicit downstream effects. To determine whether the status of these components was changed during senescence, their expression was examined in oncogene-induced and replicative senescence. Similar to THBD, thrombin, PC, and PAR1 were all significantly elevated during senescence (FIGS. 4A and 4B). Taken together, these results demonstrate that THBD signaling is up-regulated in multiple cell types by a broad-range of senescent stimuli.

### Example 3—THBD Signaling Regulates the Switch Between Senescence and Apoptosis During Oncogenic Stress and Maintains Senescent Cell Viability

Because THBD signaling was consistently up-regulated in multiple types of senescence, it was believed that THBD might have a conserved and essential function during senescence. Experiments were conducted to determine whether THBD signaling was necessary for cellular senescence initiation and establishment. To test this, IMR-90 cells were infected with lentivirus stably carrying a doxycycline-inducible (Tet-On) vector expressing oncogenic H-Ras with a scrambled control (NTC) or short-hairpin RNAs against THBD (shTHBD). After puromycin selection, cells were treated with doxycycline (dox) for 7 days to induce senescence. Cells were then stained for SA-βgal and immunoblotted for senescent markers, pRB, p21, and p16.

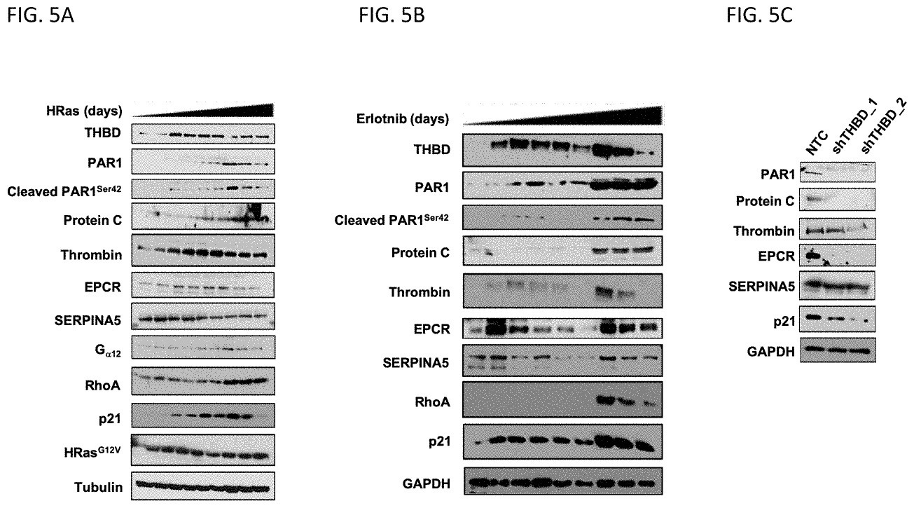

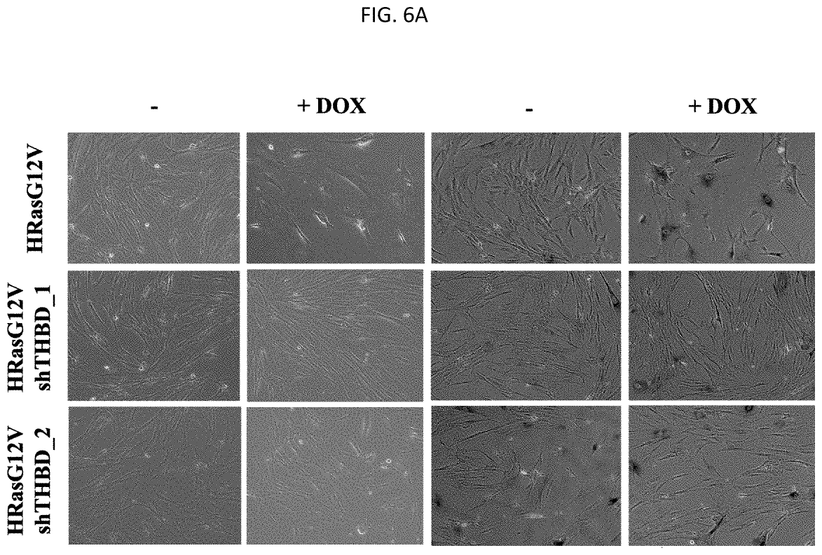

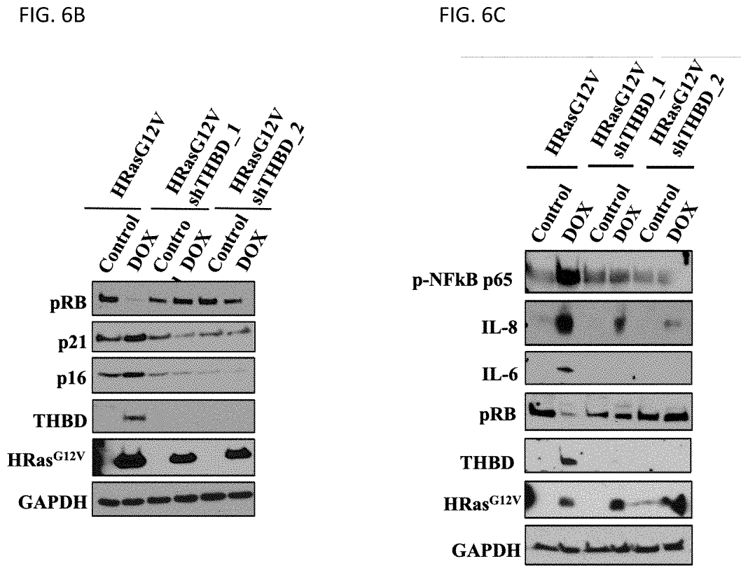

Control cells exhibited a noticeable decrease in proliferation, enlarged and flattened morphology, and robust SA-βgal staining following doxycycline treatment. In contrast, THBD-knockdown cells displayed normal proliferation and morphology and exhibited minimal SA-βgal staining, suggesting that knockdown of THBD attenuates H-Ras-induced senescence (FIG. 6A). Consistent with these observations, cyclin-dependent kinase inhibitors, p21 and p16, and Rb hypophosphorylation were increased upon doxycycline treatment in control cells but not in THBD-knockdown cells (FIG. 6B).

To further investigate the relationship between THBD signaling and cellular senescence, experiments were conducted to determine whether THBD signaling regulated the senescence-associated secretory phenotype (SASP), another hallmark of cellular senescence. To test this, the expression levels of prominent SASP factors, IL-6 and IL-8 were examined, in control cells and THBD-knockdown cells upon doxycycline treatment. Both IL-6 and IL-8 were significantly elevated in control cells but displayed no change in THBD-knockdown cells (FIG. 6C).

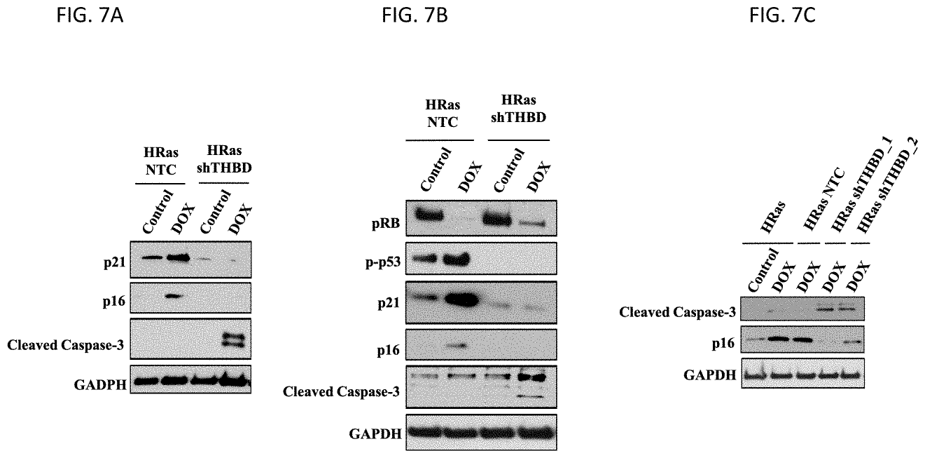

Next, experiments were conducted to determine whether knockdown of THBD affected cell proliferation and viability during continuous H-Ras overexpression. To test this, the aforementioned experiment was conducted in IMR-90 and HBE cells. Instead of treating cells with doxycycline for 7 days, treatment was extended for a longer time period. As previously observed, control cells senesced after 7 days whereas THBD-knockdown cells continued to proliferate. However, by day 11, THBD-knockdown cells began to exhibit noticeable cell rounding and detachment, suggesting apoptosis. To confirm this, the cells were harvested and immunoblotted for the apoptosis effector, cleaved caspase-3. THBD-knockdown cells exhibited a significant increase in caspase-3 cleavage whereas control cells displayed little to no increase upon doxycycline treatment (FIGS. 7A and 7B). These results suggest that THBD signaling plays an important role in cell fate determination following oncogenic stress and its up-regulation contributes to the ability of cells to bypass apoptosis in favor of senescence.

Experiments also were conducted to determine whether THBD signaling was critical for senescent cell survival. Senescence was induced in IMR-90 cell stably expressing H-Ras by treating cells with doxycycline for 7 days. Following senescence establishment, proliferating and senescent cells were infected with lentivirus carrying either non-targeting control (NTC) or short-hairpin THBD (shTHBD) and immunoblotted for cleaved caspase-3 (FIG. 7C). THBD-depletion in senescent cells resulted in a significant increase in caspase-3 cleavage whereas THBD-depletion in control cells resulted in little to no increase, indicating that THBD signaling maintains viability of oncogene-induced senescent cells.

### Example 4—THBD Signaling Regulates the Switch Between Senescence and Apoptosis During Palbociclib Treatment and Maintains Senescent Cell Viability

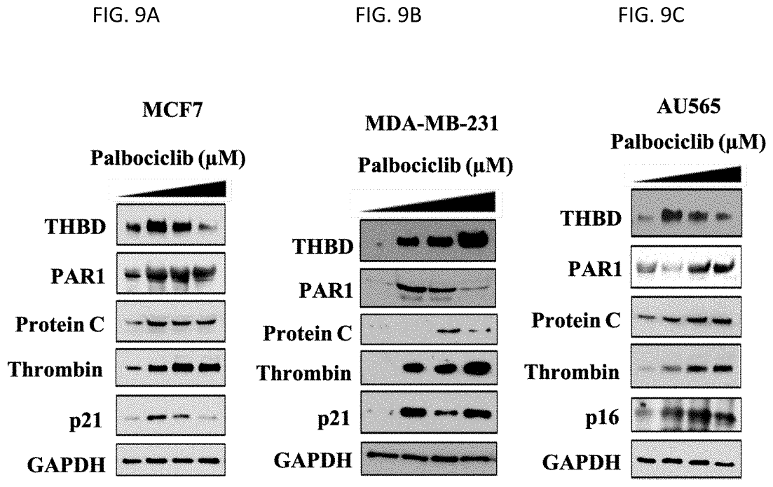

Because THBD signaling was up-regulated by numerous senescence stimuli, experiments were conducted to determine whether it was likewise up-regulated upon palbociclib treatment. MCF7, MDA-MB-231, and AU565 cells were treated with increasing doses of palbociclib for 7 days to induce senescence. Subsequent western blot analysis revealed that THBD and its signaling components, including PAR1, PC, and thrombin, were up-regulated in a dose-dependent manner (FIGS. 9A-9C).

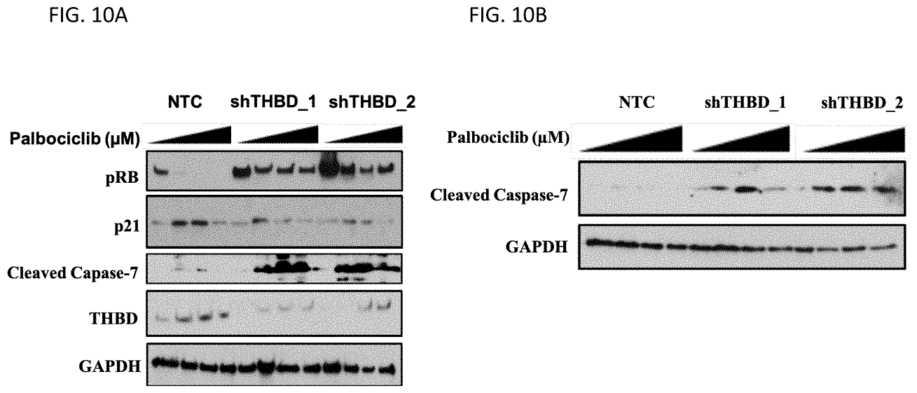

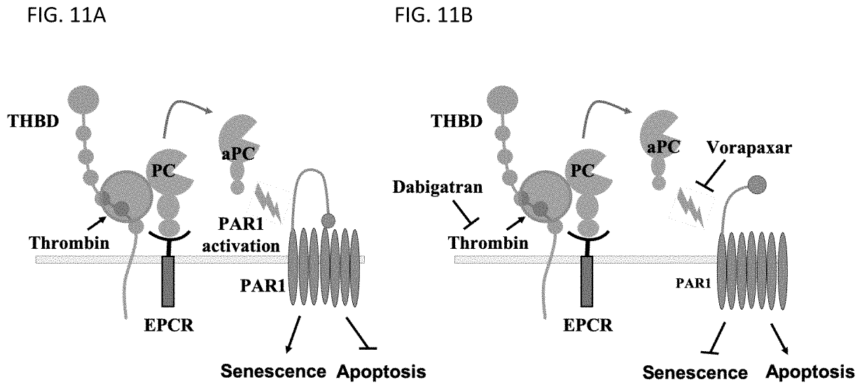

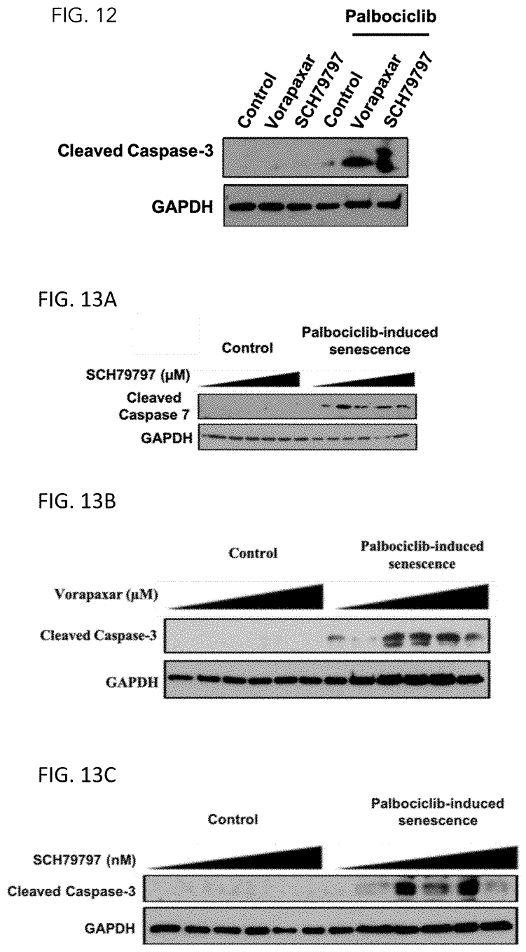

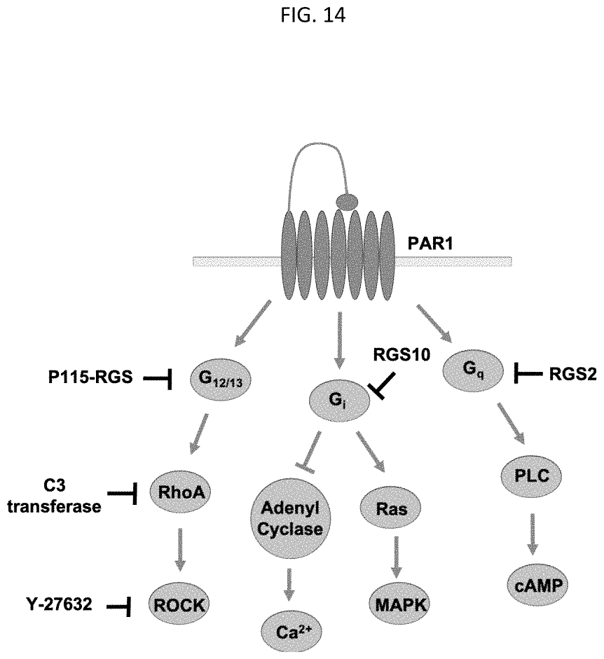

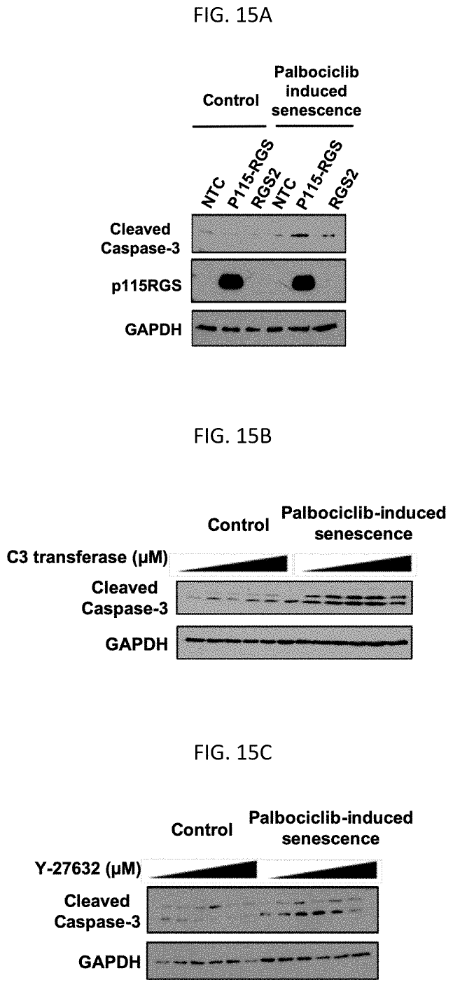

To test the functional significance of THBD signaling during palbociclib-induced senescence, MCF7 cells were infected with lentivirus carrying a scrambled control (NTC) or shRNA mixes targeting THBD. Following infection and stable selection, cells were treated with increasing doses of palbociclib and immunoblotted for senescent markers. As previously seen, control cells displayed a dose-dependent decrease in Rb phosphorylation and increase in p21 in response to palbociclib. However, THBD-knockdown cells displayed a noticeable rescue in Rb phosphorylation and minimal change in p21 levels, and a significant increase in caspase-7 cleavage (FIG. 10A).

Next, experiments were conducted to determine whether THBD exerted pro-survival functions in established senescent cells. To address this, senescence was induced in MCF7 by treating them with palbociclib. Following senescence establishment, proliferating and senescent cells were either treated with scrambled control (NTC) or shRNA (shTHBD) mixes targeting THBD. Importantly, knockdown of THBD in senescent cells resulted in noticeable cell death as indicated by an increased caspase-7 cleavage (FIG. 10B), whereas the viability of control cells was not affected by THBD knockdown. Collectively, these results indicate that THBD signaling has a multifaceted role during palbociclib-induced senescence. First, it is required for palbociclib-induced senescence as depletion of THBD drives breast cancer cells towards apoptosis rather than senescence during palbociclib treatment. Second, THBD signaling is important for senescent cell survival as THBD depletion results in significant senescent cell death.

### Example 5—Pharmacological Inhibition of PAR1 Promotes Apoptosis of Palbociclib-Induced Senescent Breast Cancer Cells

Although THBD depletion promotes apoptosis in palbociclib-induced senescent breast cancer cells, there are currently no specific inhibitors against THBD. Therefore, currently using THBD as a senolytic target may not be a feasible strategy. There are, however, inhibitors against other components of the THBD signaling pathway, including PAR1 and thrombin. Several of these inhibitors are currently used clinically for the treatment of non-cancer related diseases, such as vorapaxar (SCH530348, trade name Zontivity®, Aralez Pharmaceuticals) (Lee, C. J. and J. E. Ansell, Direct thrombin inhibitors. Br J Clin Pharmacol, 2011. 72(4): p. 581-92; Rhea, J. M. and R. J. Molinaro, Direct thrombin inhibitors: clinical uses, mechanism of action, and laboratory measurement. MLO Med Lab Obs, 2011. 43(8): p. 20, 22, 24; Gurbel, P. A., et al., Vorapaxar: a novel PAR1 inhibitor. Expert Opin Investig Drugs, 2011. 20(10): p. 1445-53; Li, Y., et al., Interference with Protease-activated Receptor 1 Alleviates Neuronal Cell Death Induced by Lipopolysaccharide-Stimulated Microglial Cells through the PI3K/Akt Pathway. Sci Rep, 2016. 6: p. 38247; Mao, X. and M. R. Del Bigio, Interference with protease-activated receptor 1 does not reduce damage to subventricular zone cells of immature rodent brain following exposure to blood or blood plasma. J Negat Results Biomed, 2015. 14: p. 3).

Vorapaxar is an oral, reversible thrombin receptor antagonist that selectively antagonizes PAR1 to prevent thrombin-related platelet activation. It is FDA-approved for the reduction of thrombotic cardiovascular events in patients with a history of myocardial infraction or peripheral arterial disease (Waasdorp, M., et al., Vorapaxar treatment reduces mesangial expansion in streptozotocin-induced diabetic nephropathy in mice. Oncotarget, 2018. 9(31): p. 21655-21662; Morrow, D. A., et al., Vorapaxar in the secondary prevention of atherothrombotic events. N Engl J Med, 2012. 366(15): p. 1404-13). Accordingly, experiments were conducted to determine whether pharmacological inhibition of PAR1 resulted in senescent cell death.

Proliferating and palbociclib-induced senescent MDA-MB-231 cells were treated with increasing doses of vorapaxar. Following treatment, cells were immunoblotted for cleaved caspase-3. Senescent MDA-MB-231 cells exhibited a dose-dependent increase in caspase-3 cleavage whereas proliferating cells exhibited a minimal increase following vorapaxar treatment (FIG. 13B). Collectively, these results indicate that inhibition of PAR1 through vorapaxar administration represents a novel therapeutic strategy for the elimination of senescent cells and attenuation of palbociclib resistance, ultimately impacting disease progression and tumor recurrence.

Lung Cancer

Dysregulation of cyclin-dependent kinases (CDKs) occurs in more than 70% of non-small cell lung cancer (NSCLC) cases and results in aberrant cell cycle control and tumorigenesis (Liu, M., et al., Mechanisms of the CDK4/6 inhibitor palbociclib (PD 0332991) and its future application in cancer treatment (Review). Oncol Rep, 2018. 39(3): p. 901-911; Nie, H., et al., Palbociclib overcomes afatinib resistance in non-small cell lung cancer. Biomed Pharmacother, 2019. 109: p. 1750-1757). Thus, there is a significant interest in investigating the role of CDK inhibitors in lung cancer. Palbociclib is the first CDK4/6 inhibitor to be clinically approved for cancer therapy and is currently used in combination with the aromatase inhibitor, letrozole, for the treatment of ER+/HER2 metastatic breast cancer (Finn, R. S., et al., Palbociclib and Letrozole in Advanced Breast Cancer. N Engl J Med, 2016. 375(20): p. 1925-1936; Finn, R. S., et al., Efficacy and safety of palbociclib in combination with letrozole as first-line treatment of ER-positive, HER2-negative, advanced breast cancer: expanded analyses of subgroups from the randomized pivotal trial PALOMA-1/TRIO-18. Breast Cancer Res, 2016. 18(1): p. 67).

In multiple phase II clinical trials investigating palbociclib as a monotherapy for NSCLC, palbociclib produced stable disease rather than partial or complete responses and led to adverse side effects, including neutropenia and leukopenia, suggesting that palbociclib therapy need to be further improved for enhanced efficacy and reduced toxicity (Nie, H., et al., Palbociclib overcomes afatinib resistance in non-small cell lung cancer. Biomed Pharmacother, 2019. 109: p. 1750-1757; Gopalan, P. K., et al., CDK4/6 inhibition stabilizes disease in patients with p16-null non-small cell lung cancer and is synergistic with mTOR inhibition. Oncotarget, 2018. 9(100): p. 37352-37366; Endelman M J, R. M., Alkbain K S, et al., A phase II study of palbociclib (P) for previously treated cell cycle gene alteration positive patients (pts) with stage IV squamous cell lung cancer (SCC): Lung-MAP sub-study SWOG 51400C. J Clin Oncol, 2017. 35: p. 1). Palbociclib achieves its therapeutic effect by inducing tumor cell senescence, a state of irreversible cell cycle arrest (Campisi, J., et al., Cellular senescence, cancer and aging: the telomere connection. Exp Gerontol, 2001. 36(10): p. 1619-37; Campisi, J., Cellular Senescence, Aging and Cancer. Scientific World Journal, 2001. 1: p. 65; Campisi, J., Cancer, aging and cellular senescence. In Vivo, 2000. 14(1): p. 183-8).

Despite being growth arrested, senescent cells remain metabolically active and have the potential to create a pro-tumorigenic microenvironment, resulting in reduced therapeutic efficacy and disease recurrence (McHugh, D. and J. Gil, Senescence and aging: Causes, consequences, and therapeutic avenues. J Cell Biol, 2018. 217(1): p. 65-77; Watanabe, S., et al., Impact of senescence-associated secretory phenotype and its potential as a therapeutic target for senescence-associated diseases. Cancer Sci, 2017. 108(4): p. 563-569; Guan, X., et al., Stromal Senescence by Prolonged CDK4/6 Inhibition Potentiates Tumor Growth. Mol Cancer Res, 2017. 15(3): p. 237-249; Capparelli, C., et al., CDK inhibitors (p16/p19/p21) induce senescence and autophagy in cancer-associated fibroblasts, “fueling” tumor growth via paracrine interactions, without an increase in neo-angiogenesis. Cell Cycle, 2012. 11(19): p. 3599-610).

Based on previous findings, a novel hypothesis was formulated that disruption of the senescence program induced by palbociclib can cause a change in cell fate from senescence to apoptosis in NSCLC cells (Alexander, P. B., et al., EGF promotes mammalian cell growth by suppressing cellular senescence. Cell Res, 2015. 25(1): p. 135-8; Chong, M., et al., CD36 initiates the secretory phenotype during the establishment of cellular senescence. EMBO Rep, 2018. 19(6); Yuan, L., et al., Switching off IMMP2L signaling drives senescence via simultaneous metabolic alteration and blockage of cell death. Cell Res, 2018. 28(6): p. 625-643). Thus, the use of senolytic therapies to promote synthetic lethality can bypass the negative side effects of senescence and enhance the efficacy of palbociclib by driving palbociclib-treated cells toward apoptosis rather than senescence.

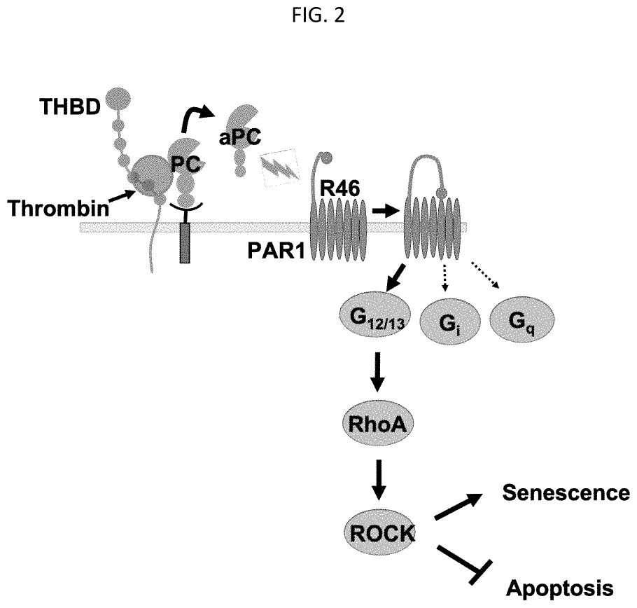

From transcriptome profiling of senescent human lung epithelial cells, thrombomodulin (THBD) was identified as a novel senolytic target for palbociclib-induced senescence. THBD is an anticoagulant receptor, mainly expressed by endothelial cells, that functions by physically binding the serine protease thrombin (Martin, F. A., et al., Thrombomodulin and the vascular endothelium: insights into functional, regulatory, and therapeutic aspects. Am J Physiol Heart Circ Physiol, 2013. 304(12): p. H1585-97; Cheng, Y., et al., Intraovarian thrombin and activated protein C signaling system regulates steroidogenesis during the periovulatory period. Mol Endocrinol, 2012. 26(2): p. 331-40; Tsiang, M., et al., Functional domains of membrane-bound human thrombomodulin. EGF-like domains four to six and the serine/threonine-rich domain are required for cofactor activity. J Biol Chem, 1992. 267(9): p. 6164-70). When bound by THBD, thrombin initiates a proteolytic cascade involving protein C, ultimately resulting in activation of the protease-activated receptor 1 (PAR1) and intracellular signal transduction (FIG. 2) (Wolter, J., et al., Thrombomodulin-dependent protein C activation is required for mitochondrial function and myelination in the central nervous system. J Thromb Haemost, 2016. 14(11): p. 2212-2226).

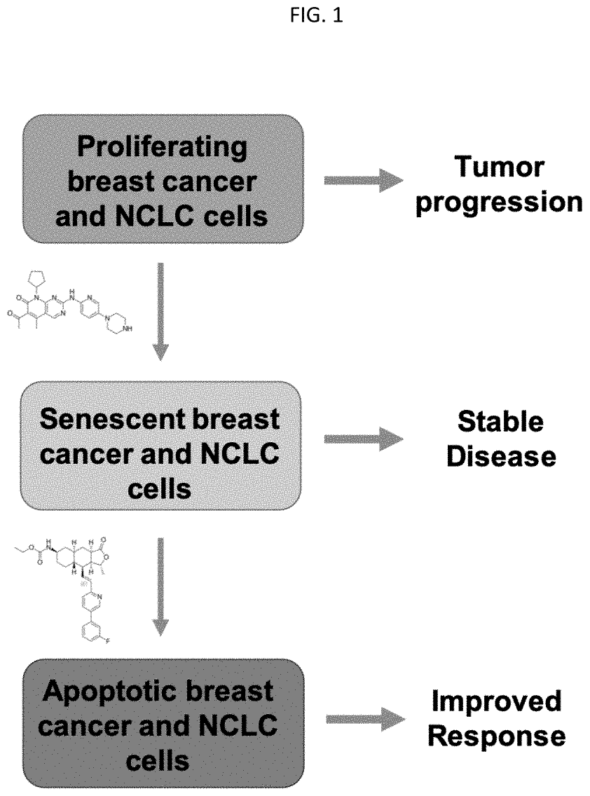

While the role of THBD as a potent anticoagulant is well documented, its role in mediating cell fate is largely undefined. Data herein demonstrate that all major components of the THBD signaling cascade are rapidly upregulated in response to senescent stimuli in NSCLC cells. Depletion of THBD or suppression of its downstream signaling in senescent cells leads to tumor cell apoptosis. Therefore, THBD signaling is a cell fate-determination pathway for senescence or apoptosis in response to palbociclib. Importantly, the THBD pathway is readily druggable, as inhibiting PAR1 with the FDA-approved drug vorapaxar causes NSCLC cells to undergo apoptosis upon palbociclib treatment. Taken together, these findings show that THBD-signaling is a novel therapeutic target that can be exploited pharmacologically to induce synthetic lethality in palbociclib-treated NSCLC. As illustrated in FIGS. 1 and 2, this hypothesis was tested with the following two aims: to dissect molecular mechanisms by which THBD signaling regulates NSCLC cell fate; and to determine the preclinical efficacy of a therapeutic strategy combining THBD axis inhibition and palbociclib against NSCLC.

The demonstrated success of palbociclib in patients with metastatic ER+/HER2− breast cancer led to the investigation of CDK inhibition as a therapeutic strategy for non-small cell lung carcinoma (NSCLC). Multiple phase II clinical trials have shown that, while palbociclib stabilizes tumor progression in patients, it fails to produce adequate response rates (Gopalan, P. K., et al., CDK4/6 inhibition stabilizes disease in patients with p16-null non-small cell lung cancer and is synergistic with mTOR inhibition. Oncotarget, 2018. 9(100): p. 37352-37366). The disease stabilization caused by this CDK4/6 inhibitor is currently thought to result from its ability to induce stable cell cycle arrest (senescence) rather than programmed cell death (apoptosis) in tumor cells (Vijayaraghavan, S., et al., CDK4/6 and autophagy inhibitors synergistically induce senescence in Rb positive cytoplasmic cyclin E negative cancers. Nat Commun, 2017. 8: p. 15916; Valenzuela, C. A., et al., Palbociclib-induced autophagy and senescence in gastric cancer cells. Exp Cell Res, 2017. 360(2): p. 390-396; Yoshida, A., E. K. Lee, and J. A. Diehl, Induction of Therapeutic Senescence in Vemurafenib-Resistant Melanoma by Extended Inhibition of CDK4/6. Cancer Res, 2016. 76(10): p. 2990-3002). Studies indicate that targeting the THBD signaling pathway provides a plausible synergistic strategy to enhance the clinical efficacy of palbociclib monotherapy. Specifically, combining THBD signaling inhibition with palbociclib can block palbociclib-induced senescence and drive NSCLC cells towards apoptosis.

Herein, multiple signaling components of the THBD pathway are shown to be upregulated during palbociclib-induced senescence. These include PAR1, protein C, and thrombin. Importantly, drugs targeting PAR1 and thrombin have already been developed for clinical applications for various non-cancer related diseases including myocardial infarction, peripheral arterial disease, and acute deep vein thrombosis. The use of these clinically-ready inhibitors will circumvent the lengthy process of drug development, resulting in shorter times to achieve improved therapeutic outcomes. As one example, the PAR1 inhibitor, vorapaxar, can be used in combination with palbociclib to attenuate palbociclib-induced NSCLC senescence and promote apoptosis, thereby bypassing undesirable features associated with senescence.

The finding that PAR1 is a novel senolytic target has broad clinical implications beyond tumor biology. During the previous decade senescent cells have emerged as an important contributor to physiological aging and various age-related diseases (Campisi, J., Cellular Senescence, Aging and Cancer. Scientific World Journal, 2001. 1: p. 65; McHugh, D. and J. Gil, Senescence and aging: Causes, consequences, and therapeutic avenues. J Cell Biol, 2018. 217(1): p. 65-77; Childs, B. G., et al., Cellular senescence in aging and age-related disease: from mechanisms to therapy. Nat Med, 2015. 21(12): p. 1424-35; Campisi, J. and L. Robert, Cell senescence: role in aging and age-related diseases. Interdiscip Top Gerontol, 2014. 39: p. 45-61). This notion is supported by the finding that eliminating senescent cells can extend healthy lifespan in mice (Bussian, T. J., et al., Clearance of senescent glial cells prevents tau-dependent pathology and cognitive decline. Nature, 2018. 562(7728): p. 578-582; Chang, J., et al., Clearance of senescent cells by ABT263 rejuvenates aged hematopoietic stem cells in mice. Nat Med, 2016. 22(1): p. 78-83; Baar, M. P., et al., Targeted Apoptosis of Senescent Cells Restores Tissue Homeostasis in Response to Chemotoxicity and Aging. Cell, 2017. 169(1): p. 132-147e16).

Because THBD is upregulated in normal cells in response to various senescence stimuli, selective depletion of senescent cells through PAR1 inhibition is a viable therapeutic approach to treat age-associated diseases. Furthermore, because of its role in coagulation, targeting THBD signaling could also be beneficial to patients suffering from cancer-associated thrombosis. It is well established that venus thromboembolism (VTE) is a common complication in cancer patients such that VTE is the second most common cause of mortality and morbidity in these patients (Abdol Razak, N. B., et al., Cancer-Associated Thrombosis: An Overview of Mechanisms, Risk Factors, and Treatment. Cancers (Basel), 2018. 10(10); Sobieraj, D. M., et al., Anticoagulation for the Treatment of Cancer-Associated Thrombosis: A Systematic Review and Network Meta-Analysis of Randomized Trials. Clin Appl Thromb Hemost, 2018: p. 10760296-18800792; Elyamany, G., A. M. Alzahrani, and E. Bukhary, Cancer-associated thrombosis: an overview. Clin Med Insights Oncol, 2014. 8: p. 129-37; Thein, K. Z., et al., Cancer Associated Thrombosis: Focus on Prevention and Treatment of Venous Thromboembolism. Cardiovasc Hematol Agents Med Chem, 2016). Given that PAR1 is required for thrombin-mediated thrombosis, inhibition of PAR1 can improve the overall survival of cancer patients by attenuating cancer-associate thrombosis, in addition to inducing tumor cell apoptosis.

### Example 6—Gene Expression Profiling Uncovers Known and Novel Regulators of Senescence

After demonstrating that selective pharmacological inhibition of the epidermal growth factor receptor (EGFR) is sufficient to rapidly induce cellular senescence in a variety of normal mammalian cell types including human bronchial epithelial (HBE) cells (Alexander, P. B., et al., EGF promotes mammalian cell growth by suppressing cellular senescence. Cell Res, 2015. 25(1): p. 135-8; Chong, M., et al., CD36 initiates the secretory phenotype during the establishment of cellular senescence. EMBO Rep, 2018. 19(6); Yuan, L., et al., Switching off IMMP2L signaling drives senescence via simultaneous metabolic alteration and blockage of cell death. Cell Res, 2018. 28(6): p. 625-643; Xiang, H., et al., UHRF1 is required for basal stem cell proliferation in response to airway injury. Cell Discov, 2017. 3: p. 17019), an unbiased gene expression profiling approach was developed to identify novel drivers of senescence by identifying genes significantly altered in senescent HBE cells versus their proliferating counterparts (Yuan, L., et al., Switching off IMMP2L signaling drives senescence via simultaneous metabolic alteration and blockage of cell death. Cell Res, 2018. 28(6): p. 625-643).

As an indication that this method can detect bona fide senescence regulators, the screening procedure uncovered the interelukin-1 receptor (IL-1R) and Notch3, both of which are previously known to regulate senescence. Moreover, using this procedure, a novel role for CD36 as a scavenger receptor essential for establishing the senescence-associated secretory phenotype (SASP) via NF-kB activation was discovered (Chong, M., et al., CD36 initiates the secretory phenotype during the establishment of cellular senescence. EMBO Rep, 2018. 19(6); Cui, H., et al., Notch3 functions as a tumor suppressor by controlling cellular senescence. Cancer Res, 2013. 73(11): p. 3451-9; Orjalo, A. V., et al., Cell surface-bound IL-1alpha is an upstream regulator of the senescence-associated IL-6/IL-8 cytokine network. Proc Natl Acad Sci USA, 2009. 106(40): p. 17031-6).

The present application focuses on a potential role for THBD in regulating the senescent cell fate because it is one the most highly upregulated genes in senescent cells (Table 1) as well as the most highly upregulated cell surface receptor (Table 2), suggesting that it might have an essential function in establishing and/or maintaining the senescent phenotype.

TABLE 1
Fold Change in Top Five Altered Genes
in Senescent v. Control HBE Cells

| Gene Name | Fold Change |
|---|---|
| IVL | 3.8 |
| SPRR2B | 3.7 |
| SLC6A14 | 3.6 |
| THBD | 3.5 |
| C10orf99 | 3.5 |

TABLE 2
Fold Change of Most Highly Upregulated Cell Surface
Receptors in Senescent v. Control HBE Cells.

| Gene Name | Fold Change |
|---|---|
| THBD | 3.5 |
| NOTCH3 | 2 |
| EPHA4 | 1.9 |
| CD36 | 1.6 |
| EPHB3 | 1.5 |
| IL1R1 | 1.5 |
| GABRP | 1.5 |

### Example 7—THBD is Induced in Response to Diverse Senescence Stimuli

THBD is a type 1 transmembrane receptor that is primarily expressed on endothelial cells (Martin, F. A., R. P. Murphy, and P. M. Cummins, Thrombomodulin and the vascular endothelium: insights into functional, regulatory, and therapeutic aspects. Am J Physiol Heart Circ Physiol, 2013. 304(12): p. H1585-97; Ikezoe, T., et al., Thrombomodulin protects endothelial cells from a calcineurin inhibitor-induced cytotoxicity by upregulation of extracellular signal-regulated kinase/myeloid leukemia cell-1 signaling. Arterioscler Thromb Vasc Biol, 2012. 32(9): p. 2259-70). Its archetypal function is to attenuate the pro-coagulant functions of thrombin and shift its specificity towards protein C (PC). THBD-mediated thrombin binding results in activation of PC, which then proteolytically cleaves protease-activated receptor 1 (PAR1) to elicit intracellular signal transduction (FIGS. 1 and 2) (Martin, F. A., R. P. Murphy, and P. M. Cummins, Thrombomodulin and the vascular endothelium: insights into functional, regulatory, and therapeutic aspects. Am J Physiol Heart Circ Physiol, 2013. 304(12): p. H1585-97; Wolter, J., et al., Thrombomodulin-dependent protein C activation is required for mitochondrial function and myelination in the central nervous system. J Thromb Haemost, 2016. 14(11): p. 2212-2226; Isermann, B., et al., Activated protein C protects against diabetic nephropathy by inhibiting endothelial and podocyte apoptosis. Nat Med, 2007. 13(11): p. 1349-58; Okamoto, T., et al., Thrombomodulin: a bifunctional modulator of inflammation and coagulation in sepsis. Crit Care Res Pract, 2012. 2012: p. 614545).

While the function of THBD in coagulation is well documented, its role in senescence has not been previously elucidated. To assess the generality of the gene profiling results above, experiments were performed to determine whether THBD is also upregulated in erlotinib-induced, oncogene-induced, and replicative senescence in IMR-90 fibroblasts, a cell type commonly used for senescence studies.

As shown in FIGS. 3A-3D, 3F, and 3G, THBD expression is strongly induced in response to all senescent stimuli in IMR-90 as well as HBE cells. Consistent with these findings, THBD mRNA levels are also markedly elevated in aged murine lung (˜70-fold) and liver tissues (˜5-fold) (FIGS. 3N and 30), suggesting that THBD upregulation is physiologically relevant to the aging process.

As previously noted, THBD is known to signal through the thrombin-PC-PAR1 axis to elicit its biological effects (FIG. 2). To determine whether these core pathway components are altered during senescence, their relative expression levels in oncogene-induced and replicative senescence was examined. Similar to THBD, thrombin, PC, and PAR1 levels are all increased in senescent cells (FIGS. 4A and 4B). Moreover, like THBD, PAR1 is also elevated in aged mouse lung, liver, and muscle tissues (FIGS. 4C-4F).

To further dissect these changes in protein expression during establishment of the senescent cell fate, a time course analysis using IMR-90 cells stably expressing oncogenic HRas under the control of doxycycline was performed. Interestingly, this procedure revealed that individual THBD signaling components are upregulated at distinct stages of senescence: THBD during senescence initiation, followed by PAR1 and PC, and finally thrombin during late senescence (FIG. 5A). Together, these results demonstrate that THBD signaling is upregulated in multiple cell types in response to a wide range of senescent stimuli, with THBD itself being the first component to accumulate during the early onset of senescence.

### Example 8—THBD is Necessary for the Initiation of Cellular Senescence

Because THBD signaling is consistently upregulated in various forms of cellular senescence, it was realized that this pathway might play an important role in establishing or maintaining the senescent cell fate. To investigate this, IMR-90 cells were infected with lentivirus stably carrying a doxycycline-inducible vector expressing oncogenic HRas together with either a scrambled control (NTC) or shRNAs targeting THBD (shTHBD). Molecularly, senescent cells are known to exhibit a common set of characteristics that includes pRb hypophosphorylation, upregulation of the cyclin-dependent kinase inhibitors p16 and p21, and increased expression of the lysosomal enzyme, β-galactosidase (senescence-associated β-galactosidase, SA-βgal) (Campisi, J., Cancer, aging and cellular senescence. In Vivo, 2000. 14(1): p. 183-8; Rodier, F. and J. Campisi, Four faces of cellular senescence. J Cell Biol, 2011. 192(4): p. 547-56; Kuilman, T., et al., The essence of senescence. Genes Dev, 2010. 24(22): p. 2463-79).

Following puromycin selection, cells treated with doxycycline (dox) for 7 days to express HRas enter a senescent state, as evidenced by SA-βgal activity, reduced pRB phosphorylation, and increased expression of the senescent markers p21 and p16 (FIGS. 6A-and 6B). In contrast, at the 7-day time point, THBD-knockdown cells displayed normal proliferation and morphology with minimal SA-βgal staining, suggesting that THBD knockdown precludes the onset of HRas-induced senescence (FIG. 6A). Moreover, levels of p21, p16, and hypophosphorylated pRb remained unaltered upon dox treatment of THBD-knockdown cells (FIG. 6B).

To further investigate the relationship between THBD signaling and cellular senescence, THBD signaling was examined to determine whether it regulates the SASP, another hallmark of cellular senescence (Coppe, J. P., et al., The senescence-associated secretory phenotype: the dark side of tumor suppression. Annu Rev Pathol, 2010. 5: p. 99-118; Coppe, J. P., et al., Senescence-associated secretory phenotypes reveal cell-nonautonomous functions of oncogenic RAS and the p53 tumor suppressor. PLoS Biol, 2008. 6(12): p. 2853-68; Ghosh, K. and B. C. Capell, The Senescence-Associated Secretory Phenotype: Critical Effector in Skin Cancer and Aging. J Invest Dermatol, 2016. 136(11): p. 2133-2139). For this, the expression levels of two prominent SASP factors, IL-6 and IL-8, were examined in control and THBD-knockdown cells after dox treatment. Both IL-6 and IL-8 were found to be significantly elevated in oncogene-induced senescent cells but remained unchanged in THBD-knockdown cells (FIG. 6C), suggesting that THBD upregulation is necessary for establishing the full senescent phenotype.

### Example 9—THBD is Essential for Maintaining the Viability of Senescent Cells

To investigate the effects of sustained THBD depletion on senescent cells, THBD expression was silenced in IMR-90 and HBE cells and assessed their proliferation and viability during continuous HRas overexpression. As previously observed, control cells senesced after 7 days whereas THBD-knockdown cells continued to proliferate. By extending HRas induction for 14 days, THBD-silenced cells began to exhibit noticeable cell rounding and detachment, suggestive of apoptosis. To specifically assay apoptotic cell death, cell lysates were immunoblotted for the apoptotic effector, cleaved caspase-3. Indeed, for both IMR-90 and HBE cells, THBD silencing led to significantly increased caspase-3 cleavage, while control cells exhibited little or no apoptosis after dox treatment (FIGS. 7A and 7B). Together, these results indicate that THBD signaling plays an essential role in cell fate determination following oncogenic stress and its upregulation facilitates senescent cell viability and escape from apoptosis.

### Example 10—Palbociclib Causes Senescence in NSCLC Cells

Because aberrant CDK4/6 activation is a common event in NSCLC, palbociclib has been tested as a monotherapy in several recent clinical trials (Gopalan, P. K., et al., CDK4/6 inhibition stabilizes disease in patients with p16-null non-small cell lung cancer and is synergistic with mTOR inhibition. Oncotarget, 2018. 9(100): p. 37352-37366). In one phase 2 study utilizing response rate as an endpoint, 19 previously treated patients with advanced NSCLC were treated with palbociclib. Of these, there were no responses and only 8 patients had stable disease lasting between 4 and 10.5 months (Gopalan, et al., 2018). Similar findings were observed in a larger phase 2 clinical trial of patients with stage IV squamous cell lung cancer. In that study, of the 32 patients treated with palbociclib, only 2 exhibited partial responses and 14 exhibited stable diseases lasting on average 1.7 months (Endelman M J, et al., A phase II study of palbociclib (P) for previously treated cell cycle gene alteration positive patients (pts) with stage IV squamous cell lung cancer (SCC): Lung-MAP sub-study SWOG S1400C. J Clin Oncol, 2017. 35: p. 1). Subsequent experimental studies conducted using lung cancer cell lines have demonstrated that palbociclib's capacity to stabilize disease is largely attributable to the induction of tumor cell senescence (Endelman, et al., 2017).

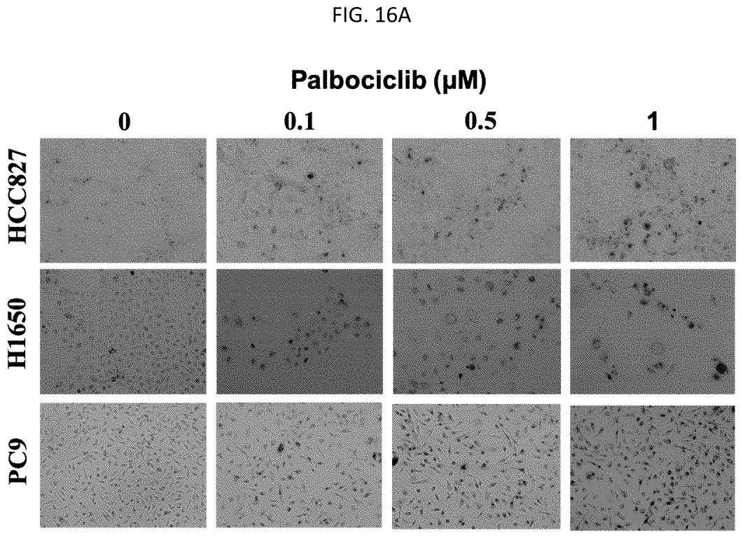

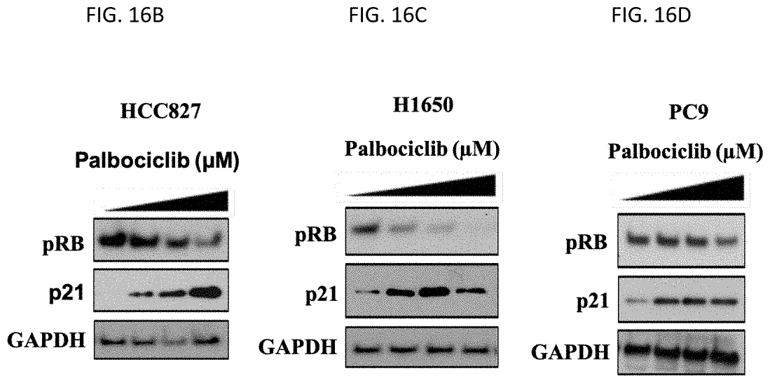

To validate the ability of palbociclib to induce cellular senescence in additional NSCLC contexts, three human lung adenocarcinoma cell lines (HCC827, HC1650, and PC9) were treated with increasing doses of palbociclib. Indeed, NSCLC cell lines treated with sub-micromolar concentrations of palbociclib exhibited cytostatic inhibition and appeared larger and flatter than untreated cells, suggesting the appearance of cellular senescence. To confirm that a senescent phenotype accounts for palbociclib-mediated growth inhibition, SA-βgal staining was conducted and, in parallel, immunoblotting for pRB, p21, and p16. All three palbociclib-treated NSCLC cells lines exhibited robust SA-βgal staining (FIG. 16A) as wells as dose-dependent increases in p21 and pRb hypophosphorylation (FIGS. 16B-16D). Overall, these results are consistent with previous reports that palbociclib impedes tumor growth primarily by inducing a senescent cell fate.

### Example 11—THBD is Essential for the Survival of Palbociclib-Treated NSCLC Cells

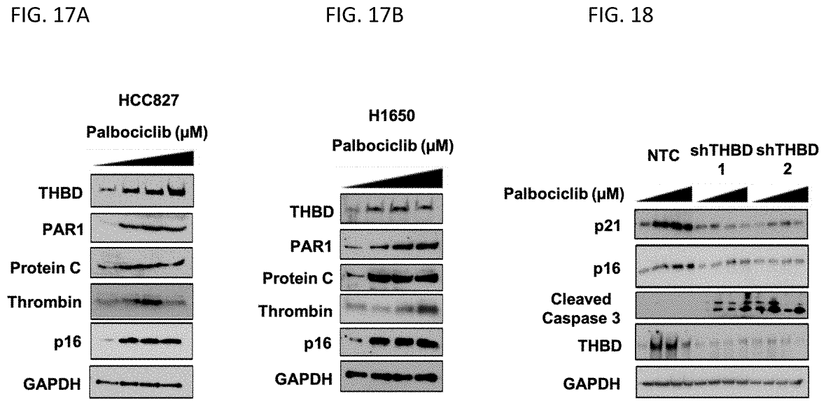

Given that THBD signaling is upregulated in response to various senescent stimuli in both IMR-90 and NHBE cells, experiments were conducted to determine whether THBD signaling is also elevated in senescent NSCLC cells. To assess this, HCC827, H1650, and PC9 cells were treated with increasing doses of palbociclib for 7 days to induce a senescent state. Subsequent western blot analysis revealed that, as in primary cells, the entire THBD signaling axis comprised of THBD, PAR1, PC, and thrombin is upregulated in senescent NSCLC cells in a dose-dependent manner (FIGS. 17 and 18A). Thus, the results herein show that THBD expression is upregulated in at least four distinct forms of cellular senescence: replicative, oncogene-induced, erlotinib-induced, and palbociclib-induced senescence.

To test the functional significance of THBD signaling induction during palbociclib-triggered senescence, HCC827 cells were infected with lentivirus carrying a scrambled control or shRNAs targeting THBD. After infection and stable selection, cells were treated with increasing doses of palbociclib and immunoblotted for senescent markers. As previously seen, control cells displayed a dose-dependent increase in p21 and p16 in response to palbociclib. In contrast, THBD-knockdown cells displayed minimal changes in p21 and p16, and strongly elevated caspase-3 cleavage (FIG. 18B). Based on these results, it was concluded that THBD signaling is strictly required to sustain the viability of NSCLC cells triggered to senesce by CDK4/6 inhibition.

### Example 12—PAR1 Inhibition Triggers the Death of Palbociclib-Treated NSCLC Cells

Studies herein demonstrate that shRNA-mediated THBD depletion induces the death of palbociclib-treated NSCLC cells. However, there are currently no selective pharmacological inhibitors targeting THBD. Therefore, focusing on THBD as a senolytic target may not be the most immediately achievable strategy. In contrast, safe and effective inhibitors already exist for two other components of the THBD axis: thrombin and PAR1. In this regard, a variety of bivalent and univalent thrombin inhibitors, as well as the selective PAR1 inhibitors SCH79797 and vorapaxar, have already been developed (discussed further in Aim 2A below) (Leonardi, S. and R. C. Becker, PAR-1 inhibitors: a novel class of antiplatelet agents for the treatment of patients with atherothrombosis. Handb Exp Pharmacol, 2012(210): p. 239-60; Liu, X., et al., Protease-activated receptor-1 (PAR-1): a promising molecular target for cancer. Oncotarget, 2017. 8(63): p. 107334-107345; Gurbel, P. A., Y. H. Jeong, and U. S. Tantry, Vorapaxar: a novel protease-activated receptor-1 inhibitor. Expert Opin Investig Drugs, 2011. 20(10): p. 1445-53; Gryka, R. J., L. F. Buckley, and S. M. Anderson, Vorapaxar: The Current Role and Future Directions of a Novel Protease-Activated Receptor Antagonist for Risk Reduction in Atherosclerotic Disease. Drugs R D, 2017. 17(1): p. 65-72).

Vorapaxar is clinically used as an oral, reversible thrombin receptor antagonist that selectively antagonizes PAR1 to prevent thrombin-dependent platelet activation (Abdulsattar, Y., T. Ternas, and D. Gar3 cia, Vorapaxar: targeting a novel antiplatelet pathway. P T, 2011. 36(9): p. 564-8). It is approved for the reduction of thrombotic cardiovascular events in patients with a history of myocardial infraction or peripheral arterial disease (Morrow, D. A., et al., Vorapaxar in the secondary prevention of atherothrombotic events. N Engl J Med, 2012. 366(15): p. 1404-1). Due to their proven safety and wide availability, PAR1 inhibitors such as vorapaxar have the potential to be rapidly repurposed for use against cancer.

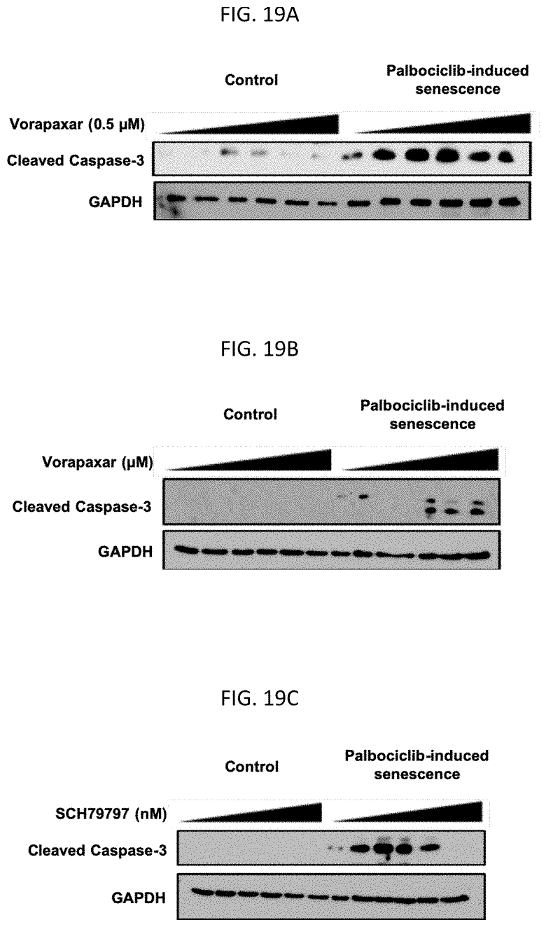

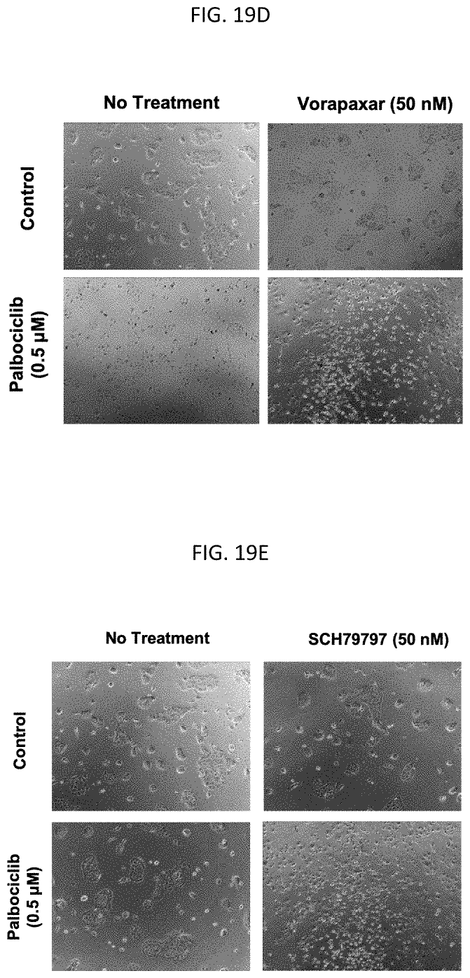

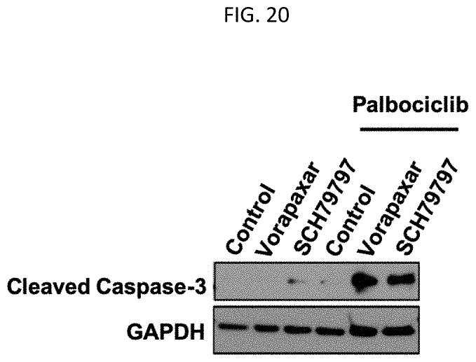

Because THBD is thought to signal primarily through PAR1, it was first tested whether, like THBD depletion, pharmacological PAR1 inhibition results in senescent cell death. For this, proliferating or palbociclib-treated senescent HCC827 and H1650 cells were treated with increasing doses of vorapaxar or SCH79797 and immunoblotted for cleaved caspase-3. Indeed, both NSCLC cell lines treated with PAR1 inhibitors exhibited a dose-dependent increase in caspase-3 cleavage (FIGS. 1(A and 19B), indicative of apoptotic cell death. Importantly, when combined with palbociclib, vorapaxar induced caspase-3 cleavage at doses as low as 25 nM, suggesting a potent synthetically lethal interaction between these compounds. Upon exposure to the palbociclib/vorapaxar or palbociclib/SCH79797 combination, HCC827 cells also exhibited noticeable cellular detachment and membrane blebbing, both of which are hallmarks of apoptosis (FIGS. 19D and 19E). None of these apoptotic features were observed when NSCLC cells were treated with any of the three compounds alone. Collectively, these results indicate that targeted PAR1 inhibition represents a novel and feasible strategy to eliminate senescent cells and improve palbociclib's therapeutic efficacy.

Taken together, these results indicate that the THBD signaling pathway represents a unique senolytic target that can be combined with palbociclib to attenuate senescence and promote synthetic lethality in NSCLC. This scientific premise is supported by discoveries herein that 1) THBD signaling is robustly upregulated in palbociclib-treated senescent NSCLC cells; 2) THBD signaling drives the senescent cell fate and is required for senescent cell viability; and 3) when combined with palbociclib, selective PAR1 inhibition attenuates senescence and induces tumor cell death.

Any patents or publications mentioned in this specification are indicative of the levels of those skilled in the art to which the invention pertains. These patents and publications are herein incorporated by reference for the indicated information to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. A method of treating a therapy-resistant cancer in a subject comprising administering to a subject a therapeutic amount of a protease-activated receptor (PAR) antagonist such that the therapy-resistant cancer is treated.

2. A method of treating a drug-induced, senescent cancer in a subject comprising administering to a subject a therapeutic amount of a protease-activated receptor (PAR) antagonist such that the drug-induced, senescent cancer is treated.

3. The method as in any one of claims 1 and 2, wherein the protease-activated receptor (PAR) antagonist is administered concurrently with one or more anti-cancer drugs.

4. The method as in any one of claims 1 and 2, wherein the protease-activated receptor (PAR) antagonist is administered prior to the administration of one or more anti-cancer drugs.

5. The method as in any one of claims 1 and 2, wherein the protease-activated receptor (PAR) antagonist is administered after the administration of one or more anti-cancer drugs.

6. The method as in any one of claims 1 and 2, wherein the subject has been treated with a therapy known to induce senescence.

7. The method of claim 6, wherein therapy is selected from the group consisting of CDK 4/6 inhibitors and DNA-damaging agents.

8. The method of claim 6, wherein the therapy is treatment with CDK 4/6 inhibitors.

9. The method of claim 3, wherein the one or more anti-cancer drugs is selected from the group consisting of palbociclib, ribociclib, and abemaciclib.

10. The method of claim 3, wherein the anti-cancer drug is palbociclib.

11. The method as in any of the preceding claims, wherein the protease-activated receptor (PAR) antagonist is a selective antagonist of protease activated receptor 1 (PAR1).

12. The method as in any of the preceding claims, wherein the protease-activated receptor (PAR) antagonist is selected from the group consisting of vorapaxar (SCH 530348), SCH 79797, atopaxar (E5555), any derivatives, esters and salts thereof, and combinations thereof.

13. The method as in any one of the preceding claims, wherein the protease-activated receptor (PAR) antagonist is vorapaxar.

14. The method of claim 13, wherein the vorapaxar is administered at a dosage of from about 0.03 mg/kg to about 15 mg/kg.

15. The method of claim 14, wherein the dosage is from about 0.5 mg/kg to about 10 mg/kg.

16. The method of claim 14, wherein the dosage is about 0.5 mg/kg, about 1 mg/kg, or about 10 mg/kg.

17. The method as in any one of the preceding claims, wherein the cancer is breast cancer.

18. The method as in any one of the preceding claims, wherein the cancer is lung cancer.

19. The method of claim 17, wherein the breast cancer is metastatic ER+, HER2− breast cancer.

20. The method of claim 17, wherein the breast cancer is a HER2+ breast cancer.

21. The method of claim 17, wherein the breast cancer is triple-negative breast cancer.

22. The method of claim 18, wherein the lung cancer is non-small cell lung cancer.

23. A method of treating a therapy-resistant cancer in a subject comprising administering to a subject a therapeutic amount of a thrombin inhibitor, such that the therapy-resistant cancer is treated.

24. A method of treating a drug-induced, senescent cancer in a subject comprising administering to a subject a therapeutic amount of a thrombin inhibitor, such that the drug-induced, senescent cancer is treated.

25. The method as in any one of claims 23 and 24, wherein the subject also suffers from cancer-associated thrombosis.

26. The method as in any one of claims 23, 24, and 25, wherein the thrombin inhibitor is administered concurrently with one or more anti-cancer drugs.

27. The method as in any one of claims 23, 24, and 25, wherein the thrombin inhibitor is administered prior to the administration of one or more anti-cancer drugs.

28. The method as in any one of claims 23, 24, and 25, wherein the thrombin inhibitor is administered after the administration of one or more anti-cancer drugs.

29. The method as in any one of claims 23, 24, and 25, wherein the subject has been treated with a therapy known to induce senescence.

30. The method of claim 29, wherein the therapy is selected from the group consisting of CDK 4/6 inhibitors and DNA damaging agents.

31. The method of claim 29, wherein the therapy is an anti-cancer drug.

32. The method of claim 31, wherein the anti-cancer drug is selected from the group consisting of palbociclib, doxorubicin, and cisplatin.

33. The method of claim 31, wherein the anti-cancer drug is palbociclib.

34. The method as in any one of claims 23, 24, and 25, wherein the thrombin inhibitor is selected from the group consisting of dabigatran, lepirudin, desirudin, bivalirudin, argatroban, any derivatives, esters and salts thereof, and combinations thereof.

35. The method as in any one of claims 23, 24, and 25, wherein the thrombin inhibitor is dabigatran.

36. The method of claim 35, wherein the dabigatran is administered in a dosage of from about 18 mg/kg to about 120 mg/kg.

37. The method of claim 37, wherein the dosage is about 18 mg/kg, about 37.5 mg/kg, about 75 mg/kg, or about 120 mg/kg.

38. The method as in any one of claims 23, 24, and 25, wherein the thrombin inhibitor is bivalirudin.

39. The method of claim 38, wherein the bivalirudin is administered in a dosage of from about 18 mg/kg to about 120 mg/kg.

40. The method of claim 39, wherein the dosage is about 18 mg/kg, about 37.5 mg/kg, about 75 mg/kg, or about 120 mg/kg.

41. A method of inducing apoptosis in a senescent tumor cell comprising administering to a subject an effective amount of a protease-activated receptor (PAR) antagonist or a thrombin inhibitor, such that apoptosis is induced in the tumor cell.

42. The method of claim 41, wherein a protease-activated receptor (PAR) antagonist is administered.

43. The method of claim 42, wherein the protease-activated receptor (PAR) antagonist is selective for PAR1.

44. The method of claim 43, wherein the protease-activated receptor (PAR) antagonist is selected from the group consisting of vorapaxar (SCH 530348), SCH 79797, atopaxar (E5555), any derivatives, esters and salts thereof, and combinations thereof.

45. The method of claim 41, wherein the protease-activated receptor (PAR) antagonist is vorapaxar.

46. The method of claim 41, wherein a thrombin inhibitor is administered.

47. The method of claim 46, wherein the thrombin inhibitor is selected from the group consisting of dabigatran, lepirudin, desirudin, bivalirudin, argatroban, any derivatives, esters and salts thereof, and combinations thereof.

48. The method of claim 46, wherein the thrombin inhibitor is dabigatran.

49. A method of inducing apoptosis in a senescent tumor cell comprising contacting the senescent tumor cell with a protease-activated receptor (PAR) antagonist or a thrombin inhibitor, such that apoptosis is induced in the tumor cell.

50. The method of claim 49, wherein the cell is contacted with a protease-activated receptor (PAR) antagonist.

51. The method of claim 50, wherein the protease-activated receptor (PAR) antagonist is selected from the group consisting of vorapaxar (SCH 530348), SCH 79797, atopaxar (E5555), any derivatives, esters and salts thereof, and combinations thereof.

52. The method of claim 50, wherein the protease-activated receptor (PAR) antagonist is selective for PAR1.

53. The method of claim 50, wherein the protease-activated receptor (PAR) antagonist is vorapaxar.

54. The method of claim 49, wherein the cell is contacted with a thrombin inhibitor.

55. The method of claim 54, wherein the thrombin inhibitor is selected from the group consisting of dabigatran, lepirudin, desirudin, bivalirudin, argatroban, any derivatives, esters and salts thereof, and combinations thereof.

56. The method of claim 54, wherein the thrombin inhibitor is dabigatran.

57. The method of claim 49, wherein the cell is characterized therapy-induced senescence.

58. The method of claim 57, wherein the therapy is selected from the group consisting of CDK 4/6 inhibitors and DNA damaging agents.

59. The method of claim 57, wherein the therapy is CDK 4/6 inhibition.

60. The method of claim 57, wherein the therapy is an anti-cancer drug.

61. The method of claim 60, wherein the anti-cancer drug is selected from the group consisting of palbociclib, ribociclib, and abemaciclib.

62. The method of claim 60, wherein the anti-cancer drug is palbociclib.
